(19) United States
(12) Patent Application Publication
(10) Pub. No.: US 2024/0156973 A1
(43) Pub. Date: May 16, 2024

(54) OSTEOTROPIC COMPOSITIONS AND USES THEREOF

(71) Applicants: Purdue Research Foundation, West Lafayette, IN (US); The Trustees of Indiana University, Bloomington, IN (US)

(72) Inventors: Philip Stewart Low, West Lafayette, IN (US); Jeffery Nielsen, Lafayette, IN (US); Melissa Ann Kacena, Zionsville, IN (US)

(21) Appl. No.: 18/547,837

(22) PCT Filed: Feb. 24, 2022
(86) PCT No.: PCT/US2022/017779

§ 371 Date: Aug. 24, 2023

Related U.S. Application Data
(60) Provisional application No. 63/153,297, filed on Feb. 24, 2021.
(60) Provisional application No. 63/180,149, filed on Apr. 27, 2021.

(51) Int. Cl. A61K 47/64; A61K 9/00; A61P 19/08

(52) U.S. Cl. CPC A61K 47/645; A61K 9/0019; A61P 19/08

(57) ABSTRACT

Bone-targeting therapeutic compounds and pharmaceutically acceptable salts thereof; pharmaceutical compositions comprising a bone-targeting therapeutic compound; processes for preparing bone-targeting therapeutic compounds; and therapeutic methods to treat bone defects.

### CROSS-REFERENCE TO RELATED APPICATIONS

This applications claims priority to U.S. Provisional Patent Application Nos. 63/153,297, filed Feb. 24, 2021; and 63/180,149, filed Apr. 27, 2021, both of which are hereby incorporated by reference as if fully set forth herein.

### STATEMENT OF GOVERNMENT SUPPORT

This invention was made with government support TR002529 awarded by the National Institutes of Health. The government has certain rights in the invention.

This invention was made with government support under BX₀₀₃₇₅₁ awarded by the Veterans Administration. The government has certain rights in the invention.

### TECHNICAL FIELD

The disclosure relates to compounds that target and treat bone defects (e.g., regenerate bone). For example, the disclosure relates to bone-targeting ligands, agents with thrombopoletic or sirtuin activity, conjugates, compositions, and methods of treating bone defects, such as bone defects resulting from bone resorption, bone trauma, osteolytic lesions (e.g., osteolytic lesions resulting from osteoporosis, cancers, and bone disease), fractures, delayed unions, and the like.

### BACKGROUND

Bone defects cause significant disability in patients. And although such bone defects can be treated (e.g., autograft surgeries using donor bone from the patient's hip may be used to correct such defects), the treatments can cause complications such as morbidity, excruciating pain, pathogen transmission, and the development of sepsis. Further, in some cases there is limited availability of donor bone. Therefore, an ongoing urgent need exists for new therapeutic interventions to treat bone defects. The present disclosure addresses this and other needs.

### SUMMARY

The disclosure relates to a seminal, far-reaching discovery of therapeutic compounds having specificity for a bone defect in a living bone of a subject and that heal the defect without adverse off-target effects. Examples of such therapeutic compounds include conjugates of the formulae I-V:

X—Y¹-Z (formula I);

X-Z (formula II);

X—X—Y¹-Z (formula III);

X—X-Z (formula IV); or

Z-Y¹—X—X—Y¹-Z (formula V)

or a pharmaceutically acceptable salt thereof, wherein:
X is a radical of a molecule having thrombopoietic activity or sirtuin activity and X—X is a dimer of a radical of a molecule having thrombopoietic activity or sirtuin activity;
Y¹, when present, is a releasable or non-releasable linker, and
Z is an osteotropic ligand.

Additional embodiments, features, and advantages of the disclosure will be apparent from the following detailed description and through practice of the disclosure.

### BRIEF DESCRIPTION OF THE DRAWINGS

FIG. 1 shows the structure of an untargeted thrombopoietin mimetic peptide (TMP).

FIG. 2 shows the structure of a targeted TMP.

FIG. 3 is a graph of saline (control) and three different daily doses (0.1, 1 and 10 nmol) of targeted TMP conjugate administered subcutaneously vs. bone volume (100 thickest micro-CT slices of fracture callus) in female Swiss Webster fracture-bearing mice (n=5) after three weeks of treatment.

FIG. 4 is a graph of saline (control) and three different daily doses (0.1, 1 and 10 nmol) of targeted TMP conjugate administered subcutaneously vs. bone volume/total volume (100 thickest micro-CT slices of fracture callus) in female Swiss Webster fracture-bearing mice (n=5) after three weeks of treatment.

FIG. 5 is a graph of saline (control) and three different daily doses (0.1, 1 and 10 nmol) of targeted TMP conjugate administered subcutaneously vs. work to fracture (mJ; total amount of energy absorbed by healed femur before refracture in post-mortem four-point bend analysis) in female Swiss Webster fracture-bearing mice (n=5) after three weeks of treatment.

FIG. 6 is a graph of saline (control) and three different daily doses (0.1, 1 and 10 nmol) of untargeted TMP administered subcutaneously vs. bone volume (100 thickest micro CTmicro-CT slices of fracture callus) in female Swiss Webster fracture-bearing mice (n=5) after three weeks of treatment.

FIG. 7 is a graph of saline (control) and three different daily doses (3.3, 33 and 330 nmol/kg) of untargeted TMP administered subcutaneously vs. callus bone volumeltotal volume in female Swiss Webster fracture-bearing mice (n=5) after three weeks of treatment.

FIG.8 is a graph of saline (control), 1 nmol/day of TMP, and 0.001 nmol/day and 0.01 nmol/day of targeted TMP conjugate administered subcutaneously vs. bone volume in female Swiss Webster fracture-bearing mice (n=5) after three weeks of treatment.

FIG. 9 is a graph of saline (control), 1 nmol/day of TMP, and 0.001 nmol/day and 0.01 nmol/day of targeted TMP conjugate administered subcutaneously vs. bone volume/total volume in female Swiss Webster fracture-bearing mice (n=5).

FIG. 10 is a graph of saline (control), 1 nmol/day of TMP, and 0.001 nmol/day and 0.01 nmol/day of targeted TMP conjugate administered subcutaneously vs. maximum (Max) load (N; maximum force healed femur withstood before refracture in post-mortem four-point bend analysis) in female Swiss Webster fracture-bearing mice (n=5).

FIG. 11 is a graph of saline, 1 nmol/day of TMP, and 0.001 nmol/day and 0.01 nmol/day of targeted TMP conjugate administered subcutaneously vs. work to fracture (mJ) in female Swiss Webster fracture-bearing mice (n=5).

FIG. 12 is a graph of saline, 1 nmol/day of TMP administered subcutaneously either every three days for three weeks or once a day for one week, either 0.01 nmol/day or 0.1 nmol/day of targeted TMP conjugate administered subcutaneously every three days for three weeks, and either 0.01 nmol/day or 0.1 nmol/day of targeted TMP conjugate administered subcutaneously once a day for one week vs. bone volume in female Swiss Webster fracture-bearing mice (n=5).

FIG. 13 is a graph of saline. 1 nmol/day of TMP administered subcutaneously either every three days for three weeks or once a day for one week, either 0.01 nmol/day or 0.1 nmol/day of targeted TMP conjugate administered subcutaneously every three days for three weeks, and either 0.01 nmol/day or 0.1 nmol/day of targeted TMP conjugate administered subcutaneously once a day for one week vs. bone volume/total volume in female Swiss Webster fracture-bearing mice (n=5).

FIG. 14A are representative μCT images of femurs 3 weeks post-surgery from 12-week-old C57BL/6 male mice that underwent a surgically created fracture and received 3×/wk injection of targeted SRT1720 (SRT1720-Aspartic Acid Oligopeptide described herein; SQ, 6 mg/kg/dose) or vehicle control.

FIG. 14B is a bar graph showing μCT analysis of the mineralized callus volume (BV/TV) for each treatment group (50, 2, 6, and 20 mg/kg/dose),

FIG. 14C is a bar graph showing μtCT analysis of the callus trabecular thickness (Tb.Th) within the callus region for each treatment group. Graph shows means±SEM and data analyzed using one-way ANOVA followed by post-hoc Newman-Keels multiple comparison testing. n=5/group. *p<0.05, compared to vehicle.

FIG. 15 is bar graphs for 12-week-old C57BL/6 male mice that underwent a surgically created fracture and received 3×/wk injection of targeted SRT1720 (SQ, 2, 6, and 20 mg/kg/dose) or vehicle control. Von Frey testing showed that 3 weeks post-surgery all mice still exhibited reduced paw withdrawal thresholds compared to baseline levels (p<0.05). However, targeted SR11720 appeared to result in improved mN forces, especially at 6 mg/kg/dose compared to vehicle control. Graph shows means±SEM and data analyzed using one-way ANOVA followed by post hoc Newman-Keels multiple comparison testing. n=5/group. *p<0.05, compared to vehicle at same time point.

FIG. 16A is representative μCT images for three-month male, C57BL/6 mice that were subjected to surgical fracture. Mice were dosed with saline or targeted TMP administered for the first week post-surgery (3.3 nmol/kg/day). Mice were euthanized 3 weeks post-surgery.

FIG. 16B are bar graphs showing mRUST scores. The four cortices are scored (1=no callus, 2-callus present, 3-bridging callus, and 4=remodeled fracture line not present) and added to give the mRUST score.

FIG. 16C is a bar graph showing toughness in torsion biomechanical testing normalized to contralateral femur (n=6-10/group, mean±SD, *p<0.05 by Student's t-test).

FIG. 16D is a bar graph showing twist-to-failure in torsion biomechanical testing normalized to contralateral femur (n=6-10,/group, mean±SD, *p<0.05 by Student's t-test).

FIG. 17 is a bar graph showing the effect of surgically induced fracture on ipsilateral weightbearing. Young (3 mo) male C57BL/6 mice were examined for static weightbearing (Bioseb) 4 days post-surgery. Weightbearing percent was significantly improved in mice with fracture targeted TMP treated mice (3.3 nmol/kg/day). Graph shows means±SEM. n=7/group. *p<0.05 via Student's t test.

FIG. 18A is images showing caspase biosensor signal present in mice subjected to unilateral femur fracture and appears to be reduced with targeted TMP treatment. Image displays bioluminescence signal present in male caspase-1 biosensor mice at left: baseline and right: 2 days post-surgery (FX). Red ovals indicate region of interest (ROI) for ipsilateral/injured (right; ipsi) and contralateral (left; contra) femurs.

FIG. 18B is a bar graph showing quantification of bioluminescence signal by IVIS imager and luminescence ratios (AUC; area under curve) in mice before (baseline or D0) and after injury (D2 and D7) post-surgery. Three-month male, C57BL/6 mice that were subjected to a surgically created fracture. Mice were dosed with saline, 33 nmol/kg/day of non-targeted TMP, or 3.3 nmol/kg/day of targeted TMP for the first week post-surgery. Mice were euthanized 2 weeks after surgery, The callus area was isolated and relative mRNA expression of PDGPβ was assessed via real-time PCR. Mean±SEM, P<0,05 as determined by repeated measures ANOVA and Tukey post-hoc test for multiple comparisons, ***P<0.001 n=5-10/group. A robust increase in caspase-1 biosensor activation was observed in the ipsilateral limb of injured mice compared to the contralateral limb irrespective of treatment. Levels were highest at D2 and while higher than baseline were dropping by D7 post-surgery. Targeted TMP treatment (3.3 nmol/kg/day, green bars) appeared to reduce caspase-1 activity in the ipsilateral limbs compared to that observed in saline treated ipsilateral limbs (red bars).

### DESCRIPTION

Before the present disclosure is further described, it is to be understood that this disclosure is not limited to the particular embodiments described, as such may, of course, vary. It is also to be understood that the terminology used is for the purpose of describing particular embodiments only, and is not intended to be limiting, since the scope of the present disclosure will be limited only by the appended claims.

Unless defined otherwise, all technical and scientific terms used have the same meaning as is commonly understood by one of ordinary skill in the art to which this disclosure belongs. All patents, applications, published applications and other publications referred to are incorporated by reference in their entireties. If a definition set forth in this section is contrary to or otherwise inconsistent with a definition set forth in a patent, application, or other publication that is incorporated by reference, the definition set forth in this section prevails over the definition incorporated by reference.

The present disclosure generally relates to thrombopoietic agents and sirtuin activators that can be incorporated into a conjugate for targeted delivery to bone to, among other things, treat bone defects (e.g., promote bone fracture healing). For example, the present disclosure is predicated, at least in part, on the discovery that a thrombopoietin mimetic peptide (TMP) or SRT1720 (sirtuin activator) can be incorporated into a conjugate for targeted delivery to bone, such as to heal bone fractures. The present disclosure is further predicated on the discovery that the sequence of TMP or SRT1720 can be improved for use in healing bone fractures, in particular by increasing megakaryocytes and by releasing growth factors during the initial inflammatory phase of fracture healing.

### Compounds

In one example, the disclosure provides a conjugate f formulae I-V:

X—Y¹-Z (formula I);

X-Z (formula II);

X—X—Y¹-Z (formula III);

X—X-Z (formula IV); or

Z-Y¹—X—X—Y¹-Z (formula V)

or a pharmaceutically acceptable salt thereof, wherein:
X is a radical of a molecule having thrombopoietic activity or sirtuin activity and X—X is a dimer of a radical of a molecule having thrombopoietic activity or sirtuin activity;
Y¹, when present, is a linker (e.g., releasable or non-releasable), and
Z is an osteotropic ligand.

The terms “osteotropic ligand” and “bone-targeting molecule,” are terms that are used interchangeably herein. Accordingly, the disclosure also relates to a compounds useful in treating, among other things, a bone defect, as the term is used herein.

An example of a compound of the formula I includes compounds of the formula Z-(reieasable linker)-X, such as a compound of the formula MP-(releasable linker)-X or AOP-(releasable linker)-X, wherein TMP represents thrombopoietin mimetic peptides, AOP represents acidic oligopeptides (e.g., oligopeptides comprising at least one, at least two, at least three, at least four, at least five, at least six, at least seven, at least eight, at least nine, at least ten, from one to eight acidic amino acids, one to five acidic amino acids, five to ten acidic amino acids or nine to 15 acidic amino acids), such as a compound of the formula:

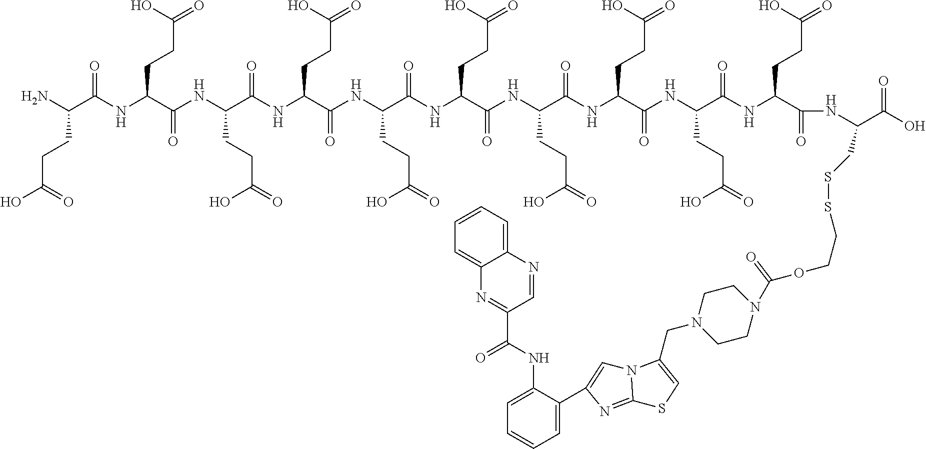 [SMILES: N[C@@H](CCC(=O)O)C(=O)N[C@@H](CCC(=O)O)C(=O)N[C@@H](CCC(=O)O)C(=O)N[C@@H](CCC(=O)O)C(=O)N[C@@H](CCC(=O)O)C(=O)N[C@@H](CCC(=O)O)C(=O)N[C@@H](CCC(=O)O)C(=O)N[C@@H](CCC(=O)O)C(=O)N[C@@H](CCC(=O)O)C(=O)N[C@@H](CCC(=O)O)C(=O)N[C@@H](CSSCCOC(=O)N1CCN(Cc2csc3nc(-c4ccccc4NC(=O)c4cnc5ccccc5n4)cn23)CC1)C(=O)O]

Another example of a compound of the formula I is a conjugate of the formula Z-PEGₚ-X, TMP-PEGₚ,-X or AOP-PEGₚ-X, wherein PEG represents a polyethylene glycol radical, TMP represents thrombopoietin mimetic peptides, AOP represents acidic oligopeptides, and p is an integer from 0 to 10.

An example of a compound of the formula V includes a conjugate of the formula Z-PEGₚ-X—X-PEG_{d}-Z, TMP-PEGₚ-X—X-PEG_{d}-TMP or AOP-PEGₚ-X—X-PEG_{d}-AOP, wherein each Z, PEG, X, Z, TMP, and AOP can be the same or different and wherein PEG represents a polyethylene glycol radical, TMP represents thrombopoietin mimetic peptides, AOP represents acidic oligopeptides, p is an integer from 0 to 10 and d is an integer from 0 to 10, such as a compound of the formula:

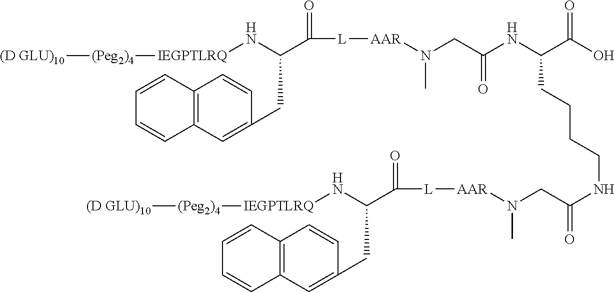

In some embodiments, provided is a conjugate of the formula X-Z, where X is a radical of a molecule comprising a sirtuin activator and Z is a molecule comprising a bone-targeting molecule, or a pharmaceutically acceptable salt thereof.

### Radical of a Molecule Having Thrombopoietic Activity or Sirtuin Activity

“Thrombopoietic activity” refers to the ability to bind to the TPO receptor (also known and c-Mpl or Mpl), activate the TPO receptor, activate downstream pathways of TPO-Mpl signaling, and/or the ability to stimulate, in vivo or in vitro, the production of platelets or platelet precursors, including but not limited to, megakaryocytes. Radicals of molecules having thrombopoietic activity include, but are not limited to, radicals of sirtuin activators and thrombopoietin mimetics, such as romiplostim, eltrombopag, oprelvekin (a recombinant interleukin 11), lusutrombopag megakaryocyte growth and development factor (MGDF), and avatrombopag radicals.

“Thrombopoietin mimetics,” “thrombopoietin radicals,” and “radical of a molecule having thrombopietic activity” refer to any compound or radical of a compound that has thrombopoietic activity. A thrombopoietin mimetic may be a peptide or a small molecule non-peptide. Thrombopoietin mimetic peptides (IMPs) comprise peptides that can be identified or derived as described in Cwirla et al. (1997), Science 276: 1696-9, U.S. Pat. No. 5,869,451; U.S. Pat. App, No, 2003/0176352, published Sep. 18, 2003; WO 03/031589, published Apr. 17, 2003, as well as WO 00/24770, published May 4, 2000, which are hereby incorporated by reference. Those of ordinary skill in the art appreciate that each of these references enables one to select different peptides than actually disclosed by following the disclosed procedures with different peptide libraries.

“Sirtuin activator,” “sirtuin activator radical,” and “radical of a molecule having sirtuin activity” refers to a compound or radical that has an ability to increase the baseline level of a sirtuin protein and/or increases the baseline level of at least one activity of a sirtuin protein relative to the activity of Sirt in the absence of the Sirt activator. In an exemplary embodiment, a sirtuin activator may increase at least one biological activity of a sirtuin protein by at least about 10%, 25%, 50%, 75%, 100%, or more. Exemplary biological activities of sirtuin proteins include deacetylation, e.g., of histones and p53; extending lifespan; decreasing apoptotic signaling, decreasing inflammation markers, modifying enzymatic activity, increasing genomic stability; regulating transcription; and controlling the segregation of oxidized proteins between mother and daughter cells. In some embodiments, the sirtuin activator is a sirtuin 1 activator. In some embodiments, the sirtuin activator is a sirtuin 3 activator.

In some embodiments, the sirtuin activator is a sirtuin 1 activator. In some embodiments, the sirtuin activator is a sirtuin 3 activator.

In some embodiments, the sirtuin activator is a small molecule sirtuin activator. In some embodiments, the sirtuin activator is a selective sirtuin activator. In some embodiments, the sirtuin activator is a non-selective sirtuin activator.

In some embodiments, the sirtuin 1 activator is SRT1720. In some embodiments, the sirtuin 1 activator is selected from the group consisting of resveratroi, forskolin, metformin, Nampt activators, AMPK activators, NMN, NAD+, STAC-5, STAC-8, STAC-9, SRT-2183, SRT1460, SRT2104, SRT3025, oxazolo[4,5-b]pyridines, pyrroio[3,2-b]quinoxalins, and combinations thereof.

In some embodiments, the sirtuin 3 activator is selected from the group consisting of 7-hydroxy-3-(4′-methoxyphenyl) coumarin (C12) and honokiol.

In some embodiments, the compounds of the formulae I-IV further comprise a second sirtuin activator where the first sirtuin activator is the same as the second sirtuin activator. In some embodiments, the sirtuin activator is not a bone anabolic compound.

“TMP” is used herein to refer to any and all peptides with thrombopoietin mimetic activity. TMP dimers are also contemplated herein. The TMP and TMP dimer can be any suitable TMP or TMP dimer, respectiveiy. For example, TMP includes a synthetic molecule with thrombopoietic activity; a TMP drier can be a stabilized dimer of a synthetic molecule with thrombopoietic activity. TMP-AF13948 and GW395058 (PMID 10437983), for example, are stabilized dimers of AF12505, a synthetic molecule that has thrombopoietic activity almost equal to intact TPO. AF12505 was discovered by directed evolution of phage, has been demonstrated to have particularly high potency and stability, and was synthesized to be used as a systemically administered drug,

Thus, the TMP can be AF12505 (IEGPTLRQWLAARA); the TMP dimer can be a modified dimer of AF12505, such as AF13948 (IEGPTLRQWLAARA-K-β-Ala-IEGPTLRQWLAARA), AF13948 in which tryptophan has been substituted with naphthalene and alanine has been substituted with sarcosine, or GW395058 (a PEGylated peptide agonist of the thrombopoietin receptor, such as described in Stem Cells 2000;18(5):380-5, which is incorporated by reference as if fully set forth herein), for example.

In at least one embodiment, X—X is a dimer of AF12505 (IEGPTLRQWLAARA), a modified dimer of AF12505, AF13948, or GW395058, Y¹ comprises (PEG₂)₄, and Z comprises ten D-glutamic acids. In an embodiment, the dimer of AF12505 is AF13948, AF13948 in which tryptophan has been substituted with naphthalene and alanine has been substituted with sarcosine, or GW395058.

In some embodiments, the thrombopoietin mimetic is a TMP. In some embodiments, the TMP is IEGPTLRQWLAARA (TPO-P). In some embodiments, the TMP is IEGPTLRQWLAARAK (TPO-P-Lys). In some embodiments, the TMP is IEGPTLROWLAARAC (TPO-P-Cys), In some embodiments, the TMP is romiplostim.

In some embodiments, the radical of a molecule having thrombopoietic activity is a thrombopoietin mimetic. In some embodiments, the thrombopoietin mimetic is an exogenous thrombopoietin mimetic.

In some embodiments, the compound includes a second molecule having thrombopoietic or sirtuin activity where the first molecule having thrombopoietic or sirtuin activity is the same as the second molecule having thrombopoietic or sirtuin activity.

In some embodiments, the thrombopoietin mimetic is a nonpeptidic thrombopoietin mimetic. In some embodiments, the nonpeptidic thrombopoietin mimetic is eltrombopag or AKR-501. In some embodiments, the nonpeptidic thrombopoietin mimetic is a conjugate where the molecule having thrombopoietic activity is not a bone anabolic compound.

It is to be understood that, when X is a dimer, i.e., X—X, each monomer (X) can be independently attached to a Y¹, such that one monomer or the other monomer is attached to a Y¹ or both monomers are attached to Y¹s, which can be the same or different. Further, each monomer, which can be independently attached to a Y¹, can be further independently attached to a Z, such that one monomer or the other monomer can be attached to Y¹ -Z or Z, wherein the Zs can be the same or different, or both monomers can be attached to Y¹ -Zs, in which the Y¹s can be the same or different and the Zs can be the same or different.

Peptide/TPO mimetics suitable for therapeutic use herein can have an IC₅₀ of about 2 mM or less (e.g., less than 1 mM, less than 500 μM, less than 250 μM, less than 1 μM, less than 500 nM, less than 250 nM, less than 1 nM, such as from about 1 nM to about 2 mM, about 100 μM to about 500 nM, about 1 nM to about 500 nM, about 500 nM to about 500 01, about 750 nM to about 750 μM or about 1 nM to about 1 μM) as determined by an assay of binding affinity for thrombopoietin receptor (TPO-R). The molecular weight of peptide mimetics suitable for therapeutic use can have a molecular weight from about 400 to about 8,000 Daltons. When derivatized, such as with a hydrophilic polymer, such as polyethylene glycol, the molecular weight can be substantially higher, such as from about 500 Daltons to about 120,000 Daltons, such as from about 8,000 Daitons to about 80,000 Daltons.

Peptide mimetics can have one or more modifications. Examples of such modifications include, but are not limited to, replacement of a peptidyl linkage with a non-peptidyl linkage, such as an alkylenecarbamate (e.g., —CH₂-carbamate, such as —CH₂OC(O)NH— and —CH₂NHC(O)O—), a phosphonate, alkyelenesulfonamide (e.g., —CH₂-sulfonamide, such as —CH₂SO₂—NH— and —CH₂NH—SO₂—), a urea, an alkylamine (e.g., —CH₂-secondary amine), or an alkylated (e.g., lower alkyl, such as C₁-C₆ alkyl) peptidyl linkage.

The N-terminus of the peptide mimetics described herein can include a derivatization. Example of such derivatizations include, but are not limited to ; —NRR¹, —NRC(O)R, —NRC(O)OR, —NRS(O)₂R, or —NHC(O)NHR, where R and R¹ are the same or different and each is independently selected from hydrogen or a lower alkyl (e.g., such as C₁-C₆ alkyl); a succinimide group; and a benzyloxycarbonyl-NH— group (e.g., such as a group having 1-3 substituents on the phenyl ring selected from lower alkyl, lower alkoxy, chloro, and bromo).

Alternatively or additionally, the C-terminus can include a derivatization. Examples of such derivatizations include, but are not limited to, —C(O)R², where R² is a lower alkoxy or NR³R⁴, where R³ and R⁴ are independently selected from hydrogen and lower alkyl (e.g., such as C₁-C₆ alkyl).

In addition, the amino acids in the TMP or dimer thereof can be L-amino acids, D-amino acids, or a combination thereof. Substitution of one or more L-amino acids with a D-amino acid can increase stability of the TMP. For example, AF13948 can be modified by changing tryptophan to naphthalene, changing alanine to sarcosine, or both.

The TMP (monomeric and dimeric) can be cyclized by the substitution of amino acid residues with cysteine residues or the insertion of cysteine residues, which can form intramoiecular disulfide bridges, which can cyclize the TMP, provided, of course, that the cyclization does not adversely affect the activity of the TMP. Cyclization also can be achieved any suitable method known in the art, e.g., by an amide bond formed between the first and last amino acids.

Examples of TMPs and dimers thereof include, but are not limited to, AF12505 (amino acid sequence=IEGPTLROWLAARA; see, e.g., Cwirla et al., Science 276: 1696-1699 (1997)) and AF13948 (dimer of AF12505). Other examples include, but are not limited to, peptide mimetics set forth in:
(1) Dower et al., Int'l Pat. App. Pub. No. WO96140750 (see, e.g., pages 7-9, 26-33, and 63-82 and the claims), published Dec. 19, 1996 peptides having a core structure comprising a sequence of amino acids:
X₁ X₂ X₃ X₄ X₅ X₆ X₇
wherein:
X₁ is C, L, M, P, Q, V;
X₂ is F, K, L, N, Q, R, S, T or V;
X₃ is C, F, I, L, M, R, S, V or W;
X₄ is any of the 20 genetically coded Lamina adds;
X₅ is A, D, E, G, K, M, Q, R, S, T, V or Y;
X₆ is C, F, G, L, M, S, V, W or Y; and
X₇ iS C, G, I, K, L, M, N, R or V;
core peptides comprising a sequence of amino adds:
X₈ G X₁ X₂ X₃ X₄X₅ W X₇
wherein:
X₁ is L, M, P, Q, or V;
X₂ is F, R, S, or T;
X₃ is F, L, V, or W;
X₄ is A, K, L, M, R, S, V, or T;
X₅ is A, E, G, K, M, Q, R, S, or T;
X₇ is C, I, K, L, M or V; and
each X₈ residue is independently selected from any of the 20 genetically coded L-amino acids, their stereoisomeric D-amino acids; and non-natural amino acids, such as where each X₈ residue is independently selected from any of the 20 genetically coded L-amino acids and their stereoisomeric D-amino acids;
core peptides wherein:
X₁ is P; X₂ is T; X₃ is L; X₄ is R; X₅ is E or Q; and X₇ is I or L;
core peptides comprising a sequence of amino acids:
X₉ X₃ G X₁ X₂ X₃ X₄ X₅ W X₇
   wherein:
X₉ is A, C, E, G, I, L , M, P, R, Q, S, T, or V; and
X₃ is A, C, D, E, K, L, Q, R, S, T, or V;
Including core peptides wherein X₉ is A or I; and X₈ is D, E, or K; and
the peptides: G G C A D G P T L R E W I S F C G G;
G N A D G P T L R Q W L E G R R P K N;
G G C A D G P T L R E W I S F C G G K;
T I K G P T L R O W L K S R E H T S;
S I E G P T L R E W L T S R T P H S,
L A I E G P T L R Q W L H G N G R D T;
C A D G P T L R E W I S F C;and
I E G P T L R Q W L A A R A;
peptides having a core structure comprising a sequence of amino adds,
C X₂ X₃ X₄ X₅ X_{g} X₇
wherein:
X₂ is K, L, N, Q, R, S, T or V;
X₃ is C, F, I, L, M, R, S or V;
X₄ is any of the 20 geneticaiiy coded Lamina adds,
X₅ is A, D, F, G, S, V or Y;
X₆ is C, F, G, L, M, S, V, W or Y; and
X₇ is C, G, I, K, L, M, N, R or V;
such as wherein:
X₄ is A, E, G, H, K, L, M, P, G, R, S, T, or W;
X₂ is S or T;
X₃ is L or R;
X₄ is R; X₅ is D, E, or G;
X₆ is F, L, or W; and
X₇ is I, K, L, R, or V;
peptides such as:
G G C T L R E W L H G G F C G G;
peptides having a structure comprising a sequence of amino adds:
X₈ C X₂ X₃ X₄ X₅ X₆ X₇;
wherein:
X₂ is F, K, L, N, Q, R, S, T or V;
X₃ is C, F, I, L, M, R, S, V or W;
X₄ is any of the 20 genetically coded L-amino acids;
X₅ is A, D, F, G, K, M, Q, R, S, T, V or Y;
X₆ is C, F, G, L, M, S, V, W or Y;
X₇ is C, G, I, K, L, M, N, R or V; and
X₈ is any of the 20 genetically coded L-amino adds, such as wherein X₈ is G, S, Y, or R;
(2) Dower et al., Int'l Pat. App. Pub. No. WO 98/25965 (see, e,g., pages 1-7, 10-13, and 50-54 and the claims), published Jun. 18, 1998

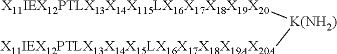

wherein
X₁₁, is hydrogen or acyl;
X₁₂ is G or sarcosine (Sar);
X₁₃ is R, A, norleucine (Nle) or N-acetyllysine (Ac-Lys);
X₁₄ is Q or E;
X₁₅ is W, L-1-naphthylalanine (1-Nal) or F;
X₁₆ is A, 5-aminopentanoic acid (Ava) or 2-aminobutyric acid (Abu);
X₁₇ is A, diphenylaianine (Diphe) or X₁₇ is absent;
X₁₈ is R, p-aminophenylalanine (p-amino-Phe), N-acetyllysine (Ac-Lys) or X₁₈ is absent;
X₁₉ and X_{19A} are the same or different and are A, βA, n-methyialanine (n-Me-Ala), sarcosine (Sar), or X₁₉ or X_{19A} is absent;
X₂₀ and X_{20A} are the same or different and are βA, or X₂₀ or X_{20A} is absent; such as compounds of the formulae:

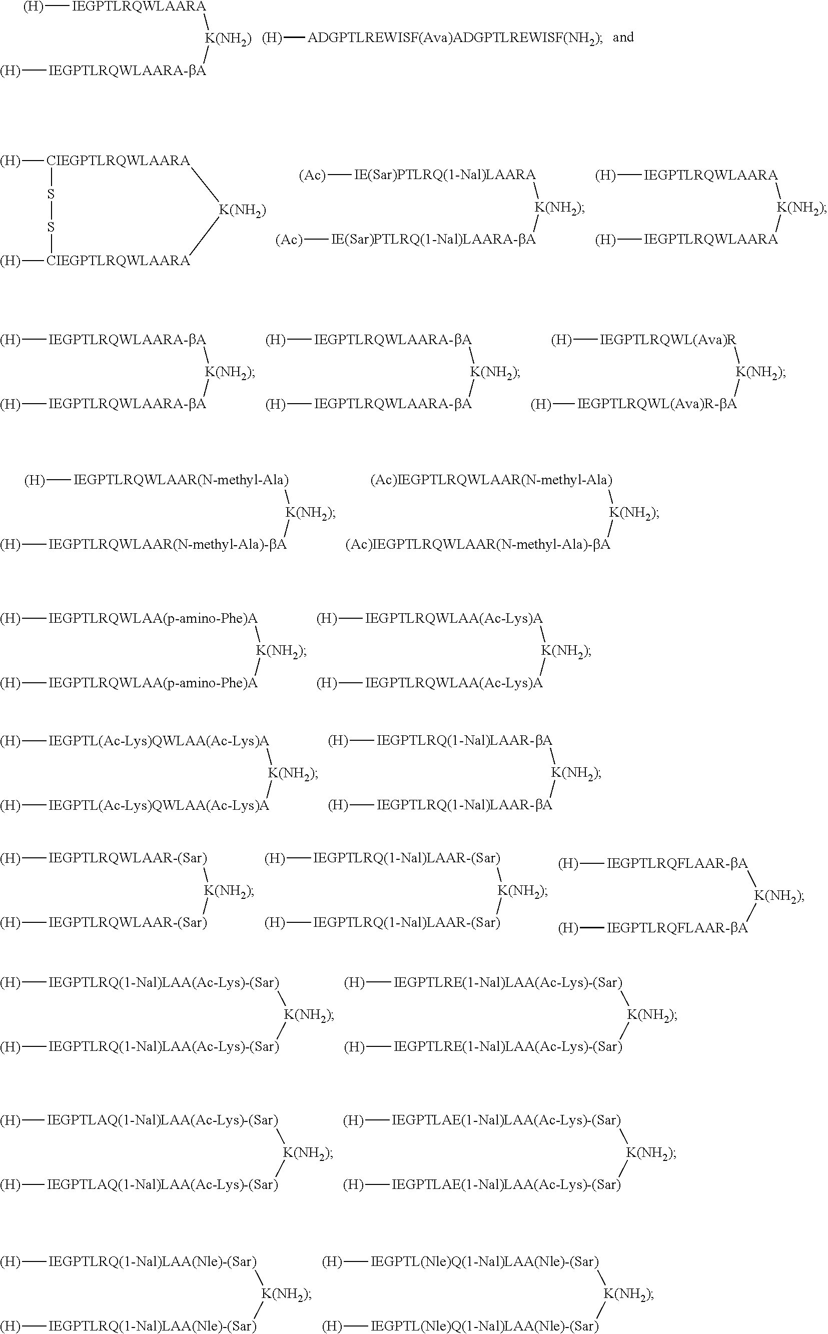

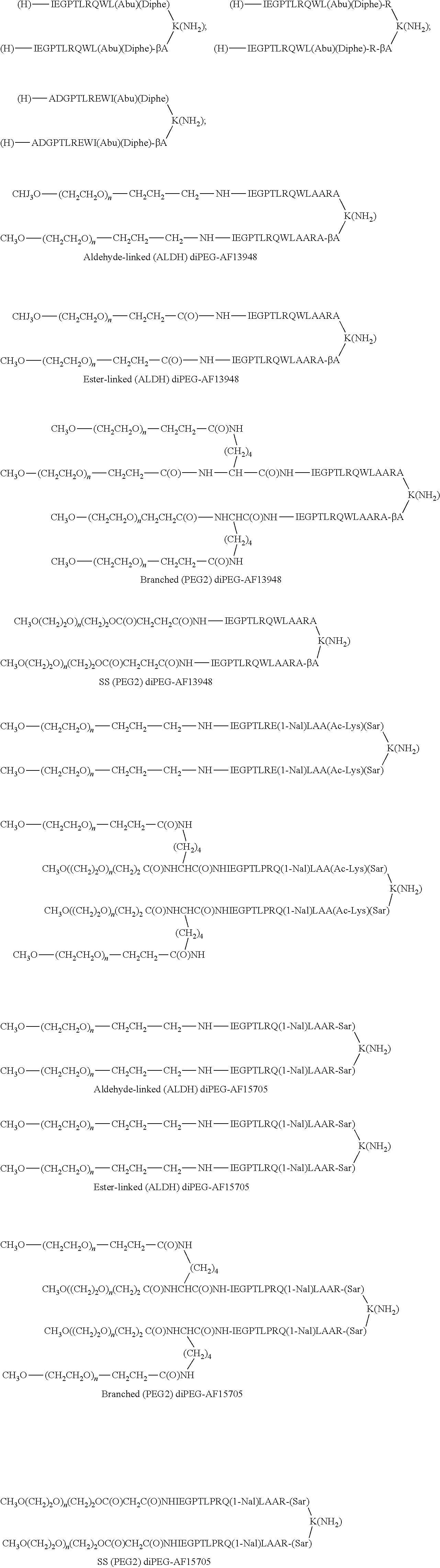

and pharmaceutically acceptable derivatives thereof, wherein the chiral amino acids are can be in the L-form and wherein “n” is an integer having a value ranging from about 5 to about 1000, e.g. 10 to about 1000, from about 100 to about 900 and, from about 110 to about 900, e.g., about 112, 225, 450 (e.g., 425-500), 675 or 900. For example, for aldehyde-linked (ALDH) PEGs, n is for example about 450, e.g., about 348-452; for ester-linked (SPA) PEGs, n is for example about 112-900, e.g., about 112, 225, 450 (e.g., 425-500), 675 or 900; for branched PEGs, n is for example about 112-450, e.g., about 112, 225 or 450; and for SS PEGs, n is for example about 112-450, e.g. about 112, 225 or 450;

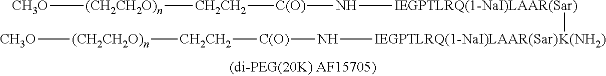

wherein n is about 450, e,g., about 425-500, and pharmaceutically acceptable derivatives thereof, wherein the chiral amino acids can be in the L-form;
and
(3) Cwirla et al, (Science 276: 1696-1699 (1997) (see, e.g., Tables 1 and 2),

TABLE 1
Thrombopoietin Receptor (TPOR) binding peptides isolated from random
peptide libraries. Amino acid sequences of clones are grouped according to
sequence homology. A consensus sequence is shown below each family (residues
conserved to a lesser extent are noted below each consensus). Highly conserved
residues are shown in bold, and cysteines are underlined. Abbreviations for the
amino acid residues are as follows:
A, Ala; C, Cys; D, Asp; E, Glu; F, Phe; G, Gly; H, His; I, Ile; K, Lys; L,
Leu; M, Met; N, Asn; P, Pro; Q, Gln; R, Arg; S, Ser; T, Thr; V, Val; W, Trp;
and Y, Tyr.

| Family 1 | Family 2A | Family 2B |
|---|---|---|
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |

[symbol]

indicates data missing or illegible when filed

TABLE 2
TPOR-binding peptides selected from random and mutagenesis peptide
libraries. Cysteine-containing peptides (AF12192 and AF12193) were
oxidized to the intramolecular disulfide-bonded (cyclic) form

| | | | |
|---|---|---|---|
| | | | IC₅₀ |
| Library | Peptide | Sequence | (nM) |

Family 1

| | | | |
|---|---|---|---|
| (X)₁₁ pVIII | AF12191 | GRVBDQIMLSLGG | 20,000 |

Family 2

| | | | |
|---|---|---|---|
| C(X)₁₀C pVIII | AF12192 | GGCTLREWLHGGFCGG | 200 |
| C(X)₁₂C pVIII | AF12193 | GGCADGPTLREWISFCGG | 60 |
| ON3398 | AF12359 | GNADGPTLRQWLEGRRPKN | 66 |
| Mutagenesis | AF12434 | LAIEGPTLRQWLHGNGPDT | 20 |
| Library* | | HGRVGPTLREWKTQVATKK | |
| ON3410 | AF12405 | TIKGFTLRQWLKSREHTS | 50 |
| Mutagenesis | | ISDGPTLKEWLSVTRGAS | |
| Library† | | STEGPTLREWLTSRTPHS | |
| AF12434 | AF12505 | IEGPTLRQWLAARA | 2 |
| Mutagenesis | | | |
| Library* | AF13948 | 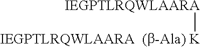 | 0.5 |

*Headpiece dimer system.
†Polysome display system.

Wherein the references (1)-(3) are all specifically incorporated herein by reference for their disclosures regarding same.

Naranda et al. (USPN 2003/0181659) discloses a TPO receptor (TPO-R) modulating oligopeptide and is specifically incorporated by reference herein for its disclosure regarding same. The oligopeptide comprises (or consists essentially of or consists of) 15-18 amino acids and has the general formula X_{A}GTLELX_{B}PX_{C}SRYRLQLX_{D}, wherein X_{A}, when present, is A, R or G, X_{B} is A or R, X_{C} is A or R, and X_{D}, when present, is RAR. Specific examples include the following:

(a)
ARGGTLELRPRSRYRL,
(b)
ARGGTLELAPASRYRL,
(c)
GTLELRPRSRYRLQL,
(d)
GTLELAPASRYRLQL,
(e)
GTLELRPRSRYRLQLR,
(f)
GTLELAPASRYRLQLR,
and
(g)
ARGGTLELRPRSRFRLQLRARLN.

The oligopeptides are disclosed to be useful for hematological disorders, such as thrombocytopenia. Naranda et al. does not disclose if such oligopeptides are useful for healing bone fractures.

Other compounds having thrombopoietic activity include, for example, a peptibody (e.g., romiplostim) or eltrombopag.

### Linker/Spacer

The terms “linker” and “spacer” are used interchangeably herein.

“Spacer” and “linker” generally refer to a filler group that may be positioned anywhere in the compounds described herein, including between the groups X and Z. The spacer may for example comprise a plurality of neutral, non-charged amino acids e.g., glycine, alanine, leucine, isoleucine, valine, proline, methionine, tryptophan, tyrosine, threonine, serine, β-alanine, γ-amino butyric acid, epsilon amino caproic acid; or PEGn (n=1-10), PPGn (n=1-6), amino-PEGn-carboxy group (n=1-6), including for example, 8-amino-3,6-dioxaoctanoic acid, 11-amino-3,6,9-trioxaundecanoic acid, and 14-amino-3, 6, 9, 12-tetraoxatetradecanoic acid, and amino-PPGn-carboxy oligomers (e.g., n=1-6). These spacers may be homogenous (e.g., all glycine, alanine, etc., or other single amino acid) or heterogeneous (e.g., more than one type of amino acid, ethylene glycol/propylene glycol, or a hybrid amino acid/amino-PEGn-carboxy or amino-PPGn-carboxy where n=1-6), and is desirably (but not by way of limitation) 3.0-21 Å (0.3 nm-2.1 nm) (or 1 to 7 amino acids) in length. The spacer may be comprised of neutral monomers comprising ethylene glycol for example, or other similar monomer units (e.g., propylene glycol), which together have a length of 3.0-21 Å (0.3 nm-2.1 nm) such that the spacer places the thrombopoietic agent or sirtuin activator in a desirable position with respect to its receptors.

Linker, Y¹, can be present, in which case it can be releasable or non-releasable. When Y¹ is non-releasable, it can contain at least one carbon-carbon bond, amide bond, carbon-oxygen bond (e.g., ether or PEG linker), and/or carbon-sulfur bond (e.g., maleimide). When Y¹ is releasable, it can contain at least one disulfide (S—S), at least one ester (O(C═O)), and/or at least one protease-specific amide bond. Y¹ can comprise (PEG₂)_{q}, wherein q=an integer of at least one. In some embodiments q=an integer from 1 to 20.

Releasable linkers also include hydrolysable linkers, such as a radical of 11-aminoundecanoic acid:

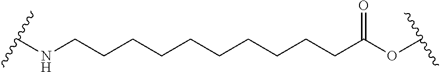

In some embodiments, the linker is positioned between two radicals of a molecules having thrombopoietic or sirtuin activity.

Releasable linkers can also comprise a disulfide moiety. In some embodiments, the releasable linker comprises the structure:

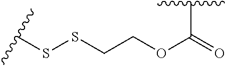 [SMILES: *SSCCOC(*)=O]

The linker Y¹ can comprises a PEG spacer, such as a PEG spacer of the formula:

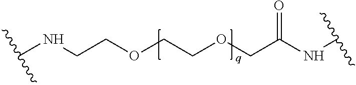

where q is an integer from 1 to 10; or

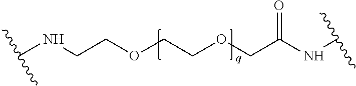

where q is an integer selected from the group consisting of 1, 2, 3, 4, 5, 6, 7, 8, 9, and 10; or a PEG spacer of the formula:

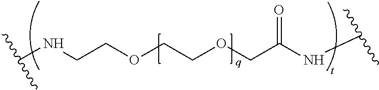

wherein q is an integer from 1 to 10 and t is an integer from 1 to 10.

The linker Y¹ can comprise a PEG₂ spacer (8-amino-3,6-dioxaoctanoic acid); a PEG₃ spacer (12-amino-4,7,10-trioxadodecanoic acid); or a PEG4 spacer (15-amino-4,7,10,13-tetraoxapenta-decanoic acid).

In some embodiments, the compound comprises a second spacer. In some embodiments, the spacer is positioned between the radical of a molecule having thrombopoietic or sirtuin activity and the osteotropic ligand or bone-targeting molecule, which are terms that are used interchangeably herein.

### Osteotropic Ligand/Bone Targeting Molecule

The osteotropic ligand can be any suitable osteotropic ligand, For example, the osteotropic ligand can access a bone marrow microenvironment.

In some embodiments, the osteotropic ligand is a bone defect targeting molecule, wherein “bone defect” is defined herein to include bone defects resulting from bone resorption, bone trauma, osteolytic lesions (e.g., osteolytic lesions resulting from osteoporosis, cancers, and bone disease), fractures, delayed unions, and the like. In some embodiments, the osteotropic ligand is a bone fracture targeting molecule, In some embodiments, the osteotropic ligand has an affinity for exposed hydroxyapatite at the fracture site.

The osteotropic ligand can, for example, comprise at least an acidic, basic, hydrophilic, hydrohobic or neutral peptide linked to an acidic peptide or nonpeptidic polyanion for use in targeting the compounds described herein (e,g., the conjugates of the formulae I-V) to a bone fracture surface, Such osteotropic ligands are described, for example, in WO2018/102616, which is incorporated by reference as if fully set forth herein. The osteotropic ligand can also be an osteotropic peptide, such as an acidic oligopeptide (AOP), an osteotropic small molecule, bisphosphonate, or tetracycline, for example. The osteotropic peptide, such as an AOP, can comprise at least 4 amino acid residues, such as 4 or more, such as 6 or more, 10 or more, 20 or more (such as from 6 up to, and including, 20), 30 or more, 40 or more, 50 or more, 75 or more, or 100 or more. The osteotropic peptide, such as an AOP, can comprise at most 100 amino acid residues, such as 100 or less, 75 or less, 50 or less, 40 or less, 30 or less, 20 or less, or 10 or less, but typically not less than 4 amino acid residues. The osteotropic peptide, such as an AOP, can comprise not less than 4 and not more than 30 amino acid residues, such as not less than 4 and not more than 20, such as from about 4 to about 20 (such as 4 to about 20 or about 4 to 20), e.g., 4, 5, 6, 7, 8, 9, 10, 11, 12, 13, 14, 15, 16, 17, 18, 19 or 20 amino acid residues. An AOP can comprise at least about 10 amino acids, such as 10 amino acids, or at least about 20 amino acids, such as 20 amino acids, such as 21, 22, 23, 24, 25, 30, 35, 40, 45, 50, 55, 60, 65, 70, 75, 80, 85, 90, 95 or as many as 100 amino acid residues. The osteotropic peptide, such as an AOP, can comprise aspartic acid (represented by the letter D), glutamic acid (represented by the letter E), aminoadipic acid, or a combination of two or more of the foregoing. The amino acids, such as acidic amino adds, such as aspartic add and glutamic add, can have D chirality, L chirality, or a mixture thereof, e.g., D-aspartic add, L-aspartic add, D-glutamic add, and/or L-glutamic add. Z can comprise not less than 4 and not more than 20 D-glutamic add residues, L-glutamic acid residues, or a combination thereof, Z can comprise not less than 4 and not more than 20 D-aspartic add residues, L-aspartic acid residues, or a combination thereof. The osteotropic peptide, such as an AOP, can be linear or branched. A linear osteotropic peptide, such as an AOP, can be superior to a branched AOP (e.g., due to a reduction in, or the absence of, interference), The osteotropic peptide, such as an AOP, can comprise one or more neutral or basic amino acids and/or one or more non-naturally occurring amino acids (e.g., provided the osteotropic peptide, such as an AOP, functions effectively as an osteotropic ligand). Examples of synthetic (or non-naturally occurring) amino acids include, but are not limited to, amino acids in which the amino group is separated from the carboxyl group by more than one carbon atom, such as β-alanine and y-aminobutyric acid, D-amino acids corresponding to naturally occurring L-amino acids, L-1-naphthyl-alanine, L-2-naphthyl-alanine, L-cyclohexylalanine, L-2-amino-isobutyric acid, a sulfoxide derivative of methionine, and a suifone derivative of methionine. Other examples of a non-naturally occurring amino acid include sarcosine, which is an N-alkylglycine, and naphthalene. The synthetic amino acids can be acidic, neutral or basic.

In some embodiments, the osteotropic ligand comprises an oligopeptide. In some embodiments, the osteotropic ligand comprises a negatively charged oligopeptide. In some embodiments, the osteotropic ligand comprises an acidic oligopeptide. In some embodiments, the osteotropic ligand comprises an acidic oligopeptide that comprises from 4 to 40 amino acids.

In some embodiments, the osteotropic ligand comprises an acidic oligopeptide comprising one or more amino acids selected from the group consisting of: D-aspartic acid, L-aspartic acid, D-glutamic acid, and L-glutamic acid. In some embodiments, the osteotropic ligand comprises deca-glutamic acid. In some embodiments, the osteotropic ligand comprises deca-aspartic acid.

In some embodiments, the compound of the formulae I-IV can further include a second bone-targeting molecule. In some embodiments, the second bone-targeting molecule is the same as the first bone-targeting molecule.

### Methods of Preparation

The conjugates described herein (e.g., the conjugates of the formulae I-V) can be prepared by conventional methods known in the art, such as standard solid phase techniques, and methods exemplified herein. Standard techniques include, for example, exclusive solid phase synthesis, partial solid phase synthesis, solid phase peptide synthesis, fragment condensation, classical solution synthesis, recombinant DNA technology, and fusion protein (with purification by affinity reagent followed by proteolytic cleavage). Dimerization methods are described in the art (see, e.g., U.S. Pat. No. 8,258,258 B2).

### Methods of Use

In some embodiments, provided is a method to treat a bone defect in a living bone in a subject, the method comprising the step of administering a therapeutically effective amount of a compound of the formula X-Z, where X comprises a radical of a molecule having thrombopoietic or sirtuin activity and Z comprises a bone-targeting molecule.

Further provided is a pharmaceutical composition comprising a therapeutically effective amount of a conjugate described herein (e.g., the conjugates of the formula I-IV or, in a method of treatment, e.g., an unconjugated TMP or unconjugated TMP dimer) and a pharmaceutically acceptable carrier or excipient. The composition can be an injectable composition, such as a composition that can be injected subcutaneously.

The therapeutically effective amount of a conjugate described herein (e.g., the conjugates of the formula I-IV, or, in a method of treatment, e.g., an unconjugated TMP or unconjugated TMP dimer) or composition comprising same can be determined in accordance with methods known in the art (e.g., animal models, human data, and human data for compounds that exhibit similar pharmacological activities). The therapeutically effective amount can be determined by taking into consideration various factors, such as the potency of the conjugate, body weight, mode of administration, and the type and location of fracture and its causation. The therapeutically effective amount can range from about 0.1 μg/kg/day, 1 μg/kg/day, 0.1 ng/kg/day, 1 ng/kg/day, 0.1 μg/kg/day, such as 0.5 μg/kg/day, 0.7 μg/kg/day, or 0.01 mg/kg/day up to about 1,000 mg/kg/day. Intravenous doses can be several orders of magnitude lower. The compound/composition can be administered more than once, such as daily (1-3 or more times per day), weekly (including 1-3 or more times on a given day), bi-weekly (including 1-3 or more times on a given day), monthly (including 1-3 or more times on a given day), or bimonthly (including 1-3 or more times on a given day).

Still further provided is a method of treating a bone fracture in a patient. The method comprises administering to the patient a therapeutically effective amount of a conjugate or a pharmaceutical composition comprising same. Administering can be by any suitable route, such as by injecting, such as injecting subcutaneously.

The ability of an AOP to deliver an attached TMP to a fracture site can involve factors such as the chemical characteristics of the TMP (including whether or not it is dimerized), the AOP side chain structure, the AOP length, AOP linearity/branching, and/or AOP stability.

Targeted delivery of a TMP localizes the TMP to the bone fracture, even when injected, such as subcutaneously, at a distal site. Targeted delivery allows for repeated administration, including repeated administration at a safe (e.g., relatively low) dose. Targeted delivery minimizes, if not eliminates, drift of the TMP into other tissues and unwanted mineralization. Bone growth can be stimulated for a longer period of time, rather than the stimulation that is only afforded upon administration during a surgical procedure.

The conjugate can be administered with one or more other active agents, whether in the same composition or separate compositions, which can be administered by the same or different routes and at the same time or different times. Examples of other active agents include, but are not limited to, vascular endothelial growth factor (VEGF), bone morphogenetic protein 2 (BMP2), bone morphogenetic protein 7 (BMP7), transforming growth factor β1 (TGFβ1), interleukin growth factor 1 (IGF1), and/or platelet-derived growth factor-BB (PDGF).

Even still further provided is another method of treating a bone fracture in a patient. The method comprises administering to the patient a therapeutically effective amount of a TMP, a dimer of TMP, or a pharmaceutical composition comprising same. The TMP or dimer thereof is AF12505 (IEGPTLRQWLAARA), a modified dimer of AF12505, AF13948, AF13948 in which tryptophan has been substituted with naphthalene and alanine has been substituted IAlith sarcosine, or GW395058 (dimer of AF12505). Administering can be injecting, such as injecting subcutaneously.

In one aspect, the present disclosure provides methods to treat a bone defect in a living bone in a subject (e.g., a vertebrate subject), the method comprising the step of:
administering a therapeutically effective amount of a compound described herein (e.g., compounds of the formulae I-V) to a subject in need thereof.

In one aspect, the present disclosure provides methods to treat a bone defect in a living bone in a subject (e.g., a vertebrate subject), the method comprising the step of: administering a therapeutically effective amount of a compound described herein (e.g., compounds of the formulae I-V) to a subject in need thereof.

In some embodiments, the bone defect comprises a fracture. In some embodiments, the bone defect comprises a segmental bone defect. In some embodiments, the bone defect comprises a delayed healing fracture. In some embodiments, the bone defect comprises a fracture and the fracture is selected from the group consisting of a displaced fracture, a non-displaced fracture, a closed fracture and an open fracture. In some embodiments, the bone defect comprises a fracture and the fracture is selected from the group consisting of an avulsion fracture, a buckled fracture, a comminuted fracture, a compression fracture, a linear fracture, oblique fracture, a pathological fracture, a stress fracture, and a transverse fracture.

In some embodiments, the bone is selected from the group consisting of a skull bone, a vertebral bone, a thoracic bone and a long bone. In some embodiments, the segmental bone is selected from the group consisting of a humerus, a radius and an ulna. In some embodiments, the segmental bone is selected from the group consisting of a femur, a tibia and a fibula.

In some embodiments, the bone healing in the bone defect is accelerated. In some embodiments, complete bone bridging results from the treatment.

In some embodiments, specificity of the compounds disclosed herein for a bone defect site is greater than 50%. In some embodiments, bone healing in the defect is accelerated and the accelerated bone healing is characterized by increased osteoblast production at the bone defect. In some embodiments, the accelerated bone healing is characterized by an increase in osteoblast production within the bone defect. In some embodiments, the accelerated bone healing is characterized by an increase in osteoclastogenesis. In some embodiments, the accelerated bone healing increases the remodeling of the bone. In some embodiments, the accelerated bone healing increases bone volume, increases the amount of new bone marrow formation, or both. In some embodiments, the accelerated bone healing increases hematopoiesis.

In some embodiments, from 75% to 100% bone bridging as measured by radiographic or computed tomography imaging is achieved. In some embodiments, from 75% to 200% or more callus volume is observed compared to vehicle treated or untreated controls as measured by radiographic, computed tomography, or histological imaging is achieved. In some embodiments, from 75% to 200% or more mineralized callus volume is observed compared to vehicle treated or untreated controls as measured by radiographic, computed tomography, or histological imaging is achieved.

In some embodiments, performance of treated bone increases, and the increased performance is equal to performance of an uninjured contralateral bone. In some embodiments, performance is measured by at least one biomechanical parameter selected from the group consisting of stiffness, strength, torque, maximal stiffness, ultimate torque, toughness, work to fracture, and maximum bad. In some embodiments, performance of treated bone is from at least 50% to at least 100% or more of the performance of an uninjured contralateral bone. In some embodiments, performance is measured by at least one biomechanical parameter selected from the group consisting of stiffness, strength, torque, maximal stiffness, ultimate torque, toughness, work to fracture, and maximum load.

In some embodiments, the bone defect is healed. In some embodiments, the healing is characterized by increasing osteociastogenesis in the bone defect. In some embodiments, the bone healing is characterized by bone tissue mineralization in the bone defect. In some embodiments, the bone healing is characterized by increasing endochondral bone formation. In some embodiments, the bone healing is characterized by increasing remodeling of the bone.

In some embodiments, the bone defect comprises a critically sized defect. In some embodiments, the bone defect is a nonunion.

In some embodiments, the molecule having thrombopoietic or sirtuin activity is provided in an amount per volume of the defect sufficient to repair the defect. In some embodiments, heterotopic bone formation does not occur during the treatment. In some embodiments, the compound of the formula X-Z is administered systemically. In some embodiments, the compound of the formula X-Z is administered systemically and does not result in off target adverse effects.

In some embodiments, the mammalian subject undergoes a surgery on the bone defect before the compound comprising the formula X-Z is administered and a drug is locally administered to the bone defect during the surgery. In some embodiments, the compound of the formula X-Z stimulates angiogenesis. In some embodiments, the compound of the formula X-Z stimulates endothelial lineage cells. In some embodiments, the compound of the formula X-Z stimulates mesenchymal lineage cells. In some embodiments, the compound of the formula X-Z stimulates endothelial lineage cells and stimulates mesenchymal lineage cells. In some embodiments, the compound of the formula X-Z stimulates hematopoietic lineage cells. In some embodiments, the compound of the formula X-Z stimulates megakaryocyte lineage cells.

In some embodiments, provided is a method to treat a bone defect in a living bone in a mammalian subject, the method comprising the step of administering a therapeutically effective amount of a compound of the formula X-Z, where X comprises a sirtuin activator and Z comprises a bone-targeting molecule.

In some embodiments, the method further comprises administering the compounds of the formulae I-V with an additional therapeutic compound. In some embodiments, the method further comprises administering the compounds of the formulae I-V with an additional therapeutic compound that is an antibiotic, a chemotherapeutic or a pain-relieving agent.

In some embodiments, the segmental bone is selected from the group consisting of a humerus, a radius, and an ulna. In some embodiments, the segmental bone is selected from the group consisting of a femur, a tibia, and a fibula. In some embodiments, the bone is selected from the group consisting of a skull bone, a vertebral bone, a thoracic bone, and a long bone.

In some embodiments, bone healing in the bone defect is accelerated. In some embodiments, complete bone bridging results from the treatment. In some embodiments, from 75% to 100% bone bridging as measured by radiographic or computed tomography imaging is achieved. In some embodiments, from 75% to 200% or more callus volume is observed compared to vehicle treated or untreated controls as measured by radiographic, computed tomography, or histological imaging is achieved. In some embodiments, from 75% to 200% or more mineralized callus volume is observed compared to vehicle treated or untreated controls as measured by radiographic, computed tomography, or histological imaging is achieved.

In some embodiments, performance of treated bone is equal to performance of an uninjured contralateral bone. In some embodiments, performance is measured by at least one biomechanical parameter selected from the group consisting of stiffness, strength, torque, maximal stiffness, ultimate torque, toughness, work to fracture, and maximum load. In some embodiments, performance of treated bone is from at least 50% to at least 100% performance of an uninjured contralateral bone. In some embodiments, performance is measured by at least one biomechanical parameter selected from the group consisting of stiffness, strength, torque, maximal stiffness, ultimate torque, toughness, work to fracture, and maximum load.

In some embodiments, specificity of the compound for the segmental bone defect site is greater than 50%. In some embodiments, the accelerated bone healing is characterized by increased osteoblast production at the bone defect. In some embodiments, the accelerated bone healing is characterized by an increase in osteoblast production within the bone defect. In some embodiments, the accelerated bone healing increases the remodeling of the bone. In some embodiments, the accelerated bone healing increases bone volume, increases the amount of new bone marrow formation, or both.

In some embodiments, the accelerated bone healing increases hematopoiesis. In some embodiments, the accelerated bone healing increases angiogenesis. In some embodiments, the accelerated bone healing is accompanied by decreased neuroinflammation. In some embodiments, the accelerated bone healing is accompanied by reduced pain or is pain-free,

In some embodiments, the bone defect is healed. In some embodiments, the bone healing is characterized by bone tissue mineralization in the bone defect. In some embodiments, the bone healing is characterized by increasing endochondral bone formation. In some embodiments, the bone healing is characterized by increasing remodeling of the bone. In some embodiments, the bone defect is a critical sized defect. In some embodiments, the bone defect is a nonunion.

In some embodiments, the sirtuin activator is provided in an amount per volume of the defect sufficient to repair the defect. In some embodiments, heterotopic bone formation does not occur during the treatment. In some embodiments, the compound of the formula X-Z is administered systemically. In some embodiments, the compound of the formula X-Z is administered systemically and does not result in off target adverse effects. In some embodiments, the mammalian subject undergoes a surgery on the bone defect before the compound comprising the formula X-Z is administered and a drug is locally administered to the bone defect during the surgery. In some embodiments, the compound of the formula X-Z stimulates endothelial lineage cells. In some embodiments, the compound of the formula X-Z stimulates mesenchymal lineage cells. In some embodiments, the compound of the formula X-Z stimulates endothelial lineage cells and stimulates mesenchymal lineage cells. In some embodiments, the compound of the formula X-Z stimulates hematopoietic lineage cells. In some embodiments, the compound of the formula X-Z stimulates megakaryocyte lineage cells.

In some embodiments, the disclosed compounds are useful for accelerating bone healing, bone growth, bone reconstruction, or bone repair in a subject. The accelerated bone healing may be in response to an acute traumatic injury, such as a partial or complete fracture or breakage of the bone. In some embodiments, the disclosed compounds may be administered in anticipation of a bone fracture, such as before a surgical procedure where a bone breakage is possible or expected. Alternatively, or additionally, the subject may suffer from, or be at increased risk of developing, a bone degenerative disease, such as osteoporosis brought on by aging or other factors (e.g., corticosteroids). An “increased risk” of bone degenerative disease is a risk that is elevated by known risk factors the subject possesses. For example, a woman who has undergone bone density imaging and is found to have sub-optimal or sub-normal levels of bone density would be at increased risk of osteopenia or osteoporosis. In other examples, genetic or demographic information may be used to identify someone of known risk. Other clinical settings in which bone heaiing or reconstruction can be delayed or impaired include, but are not limited to, diabetes and/or poor vascularity.

### Pharmaceutical Compositions and Delivery Routes

Compounds provided in this disclosure are usually administered in the form of pharmaceutical compositions. Thus, provided are pharmaceutical compositions that comprise one or more of the compounds or pharmaceutically acceptable salts, isomer, or a mixture thereof and one or more pharmaceutically acceptable vehicles selected from carriers, adjuvants, and excipients. The compounds provided may be the sole active ingredient or one of the active ingredients of the pharmaceutical compositions. Suitable pharmaceutically acceptable vehicles may include, for example, inert solid diluents and fillers, diluents, including sterile aqueous solution and various organic solvents, permeation enhancers, solubilizers, and adjuvants. Such compositions are prepared in a manner well-known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Ranker & C. T. Rhodes, Eds.).

In one aspect, provided are pharmaceutical compositions comprising a compound provided, or a pharmaceutically acceptable salt thereof, and a pharmaceutically acceptable excipient or carrier. In some embodiments, the pharmaceutical compositions comprise a therapeutically effective amount of a compound provided, or a pharmaceutically acceptable salt thereof, and a pharmaceutically acceptable excipient or carrier.

In some embodiments, the pharmaceutical compositions provided further comprise one or more (e.g., one, two, three, four, one or two, one to three, or one to four) additional therapeutic agents, or a pharmaceutically acceptable salt thereof. In some embodiments, the pharmaceutical compositions further comprise a therapeutically effective amount of the one or more (e.g., one, two, three, four, one or two, one to three, or one to four) additional therapeutic agents, or a pharmaceutically acceptable salt thereof.

In some embodiments, the one or more additional therapeutic agents that can be administered with compounds of the formulae I-V is an antibiotic, a chemotherapeutic agent and/or a pain-relieving agent, or a pharmaceutically acceptable salt of any of the foregoing, or any combinations thereof.

The pharmaceutical compositions may be administered in either single or multiple doses. The pharmaceutical compositions may be administered by various methods including, for example, rectal, buccal, intranasal and transdermal routes. In some embodiments, the pharmaceutical compositions may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Oral administration may be another route for administration of the compounds provided. Administration may be via, for example, capsule or enteric coated tablets. In making the pharmaceutical compositions that include at least one compound or pharmaceutically acceptable salts, isomer, or a mixture thereof, the active ingredient (such as a compound) is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the pharmaceutical compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose or any combinations thereof. The pharmaceutical compositions can additionally include lubricating agents such as talc, magnesium stearate, and mineral oil; wetting agents; emulsifying and suspending agents; preserving agents such as methyl and propyl hydroxy-benzoates; sweetening agents; and flavoring agents; or any combinations thereof.

The pharmaceutical compositions that include at least one compound/conjugate (wherein the term “conjugate” and “compound” is used interchangeably herein) or pharmaceutically acceptable salts, isomer, or a mixture thereof can be formulated so as to provide quick, sustained or delayed release of the active ingredient (such as a compound described in this disclosure) after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods employs transdermal delivery devices (“patches”). Such transdermal patches may be used to provide continuous or discontinuous infusion of the compounds in controlled amounts. The construction and use of transdermal patches for the delivery of pharmaceutical agents is well-known in the art. See, e.g., U.S. Pat. Nos. 5,023,252, 4,992,445 and 5,001,139. Such patches may be constructed for continuous, pulsatile, or on demand delivery of pharmaceutical agents.

For preparing solid compositions such as tablets, the principal active ingredient (e.g., the conjugates of the formulae I-V) may be mixed with a pharmaceutical excipient to form a solid pre-formulation composition containing a homogeneous mixture of a compound or pharmaceutically acceptable salts, isomer, or a mixture thereof. When referring to these pre-formulation compositions as homogeneous, the active ingredient may be dispersed evenly throughout the composition so that the composition may be readily subdivided into equally effective unit dosage forms such as tablets, pills and capsules.

The tablets or pills of the compounds described may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with materials such as shellac, cetyl alcohol, and cellulose acetate.

In one aspect, provided are kits that comprise a compound, or a pharmaceutically acceptable salt, stereoisomer, prodrug, or solvate thereof, and suitable packaging. In some embodiments, the kit further comprises instructions for use. In some embodiments, the kit comprises a compound, or a pharmaceutically acceptable salt, stereoisomer, prodrug, or solvate thereof, and a label and/or instructions for use of the compounds in the treatment of the indications, including the diseases or conditions, described in this disclosure.

In some embodiments, the kits further comprise one or more (e.g., one, two, three, four, one or two, one to three, or one to four) additional therapeutic agents, or a pharmaceutically acceptable salt thereof.

In one aspect, provided are articles of manufacture that comprise a compound or pharmaceutically acceptable salts, isomer, or a mixture thereof in a suitable container. In some embodiments, the container may be a vial, jar, ampoule, preloaded syringe, or intravenous bag.

Methods for preparing the novel compounds will be apparent to those of skill in the art with suitable procedures being described, for example, in the description and examples below.

### EXAMPLES

The following examples serve to illustrate the present disclosure, The examples are not intended to limit the scope of the claimed invention in any way.

### Example 1

This example describes peptide synthesis, conjugate formulation, and in vitro testing of biological activity in MCTC₃-E1 cells.

All peptides were synthesized according to the following protocol unless otherwise noted. Targeted peptides were synthesized using standard Fmoc (9-fluorenylmethoxycarbonyl) solid-phase synthesis using PYBOP (benzotriazol-1-yl-oxytripyrrolidinophosphonium hexafluorophosphate) and HBTU (2-(1H-benzotriazol-1-yl)-1,1,3,3-tetramethyluronium hexafluorophosphate) chemistries. Synthesis was confirmed by LCMS. The fraction that contained only pure peptide as assessed by LCMS was lyophilized and stored at −20° C.

### TMP Synthesis

Dimerized thrombopoietin mimetic peptides (TMP; FIG. 1) have been demonstrated to have essentially the same activity as the full TPO protein, and AF13946, the dimer of AF12505, has proven to have particularly high potency and stability and was synthesized to be used as a systemically administered drug. AF13948 (TMP) was synthesized according to standard Fmoc solid-phase peptide synthesis procedures. Briefly, TMP was synthesized in a solid-phase peptide synthesis vial under a stream of argon. 2-chlorotrityl resin (0.6 mmol/g) was loaded at 0.6 mmol/g with Nα,Nε-di-Fmoc-L-lysine for 60 mins in DCM and diisopropylethylamine. The resin was then capped with four washes of HPLC-grade MeOH, followed by 3 washes with DCM and DMF, consecutively. After each amino acid coupling reaction, Fmoc groups were removed by two 10-min incubations with 20% (v/v) piperidine in DMF. The resin was then washed twice with DMF prior to adding the next amino acid. Each amino acid was reacted in a three-fold excess with HBTU/NMM for 30 mins, followed by a double coupling with three-fold excess PyBOP/NMM for 30 mins. All amino acids were added according to the conditions above. Thereafter, the following peptide sequence was added onto the peptide using the solid-phase procedures listed above using the AAPPTec Focus XC automated peptide synthesizer: Ile-Glu-Gly-Pro-Thr-Leu-Arg-Gln-Npa-Leu-Ala-Ala-Arg-Sar.

Upon completion of the synthesis, the terminal Fmoc was removed using the same conditions as listed above, then the resin was washed three times with DMF, three times with DCM, and two times with methanol. The resin was then dried with argon gas, The dried resin with the peptide was cleaved using 95:2.5:2,5 trifluoroacetic acid water/triisopropylsilane for 2 hrs. The peptide was then precipitated from the cleavage solution using 10 times the volume of cold diethyl ether, The solution was spun at 2,000 rcf for 5 mins and then decanted, The pellet was then desiccated and submitted to LCMS for confirmation of synthesis. The crude peptide was dissolved in a mixture of DMF and water and then purified via preparative reversed-phase high-performance liquid chromatoaraphy. To purify the TMP, a C-18 column with a 0-5% ammonium acetate/acetonitrile mobile phase for 40 mins was used. The fraction that contained only pure TMP was assessed by LCMS and was lyophilized and stored at −20° C.

### Targeted TMP Synthesis

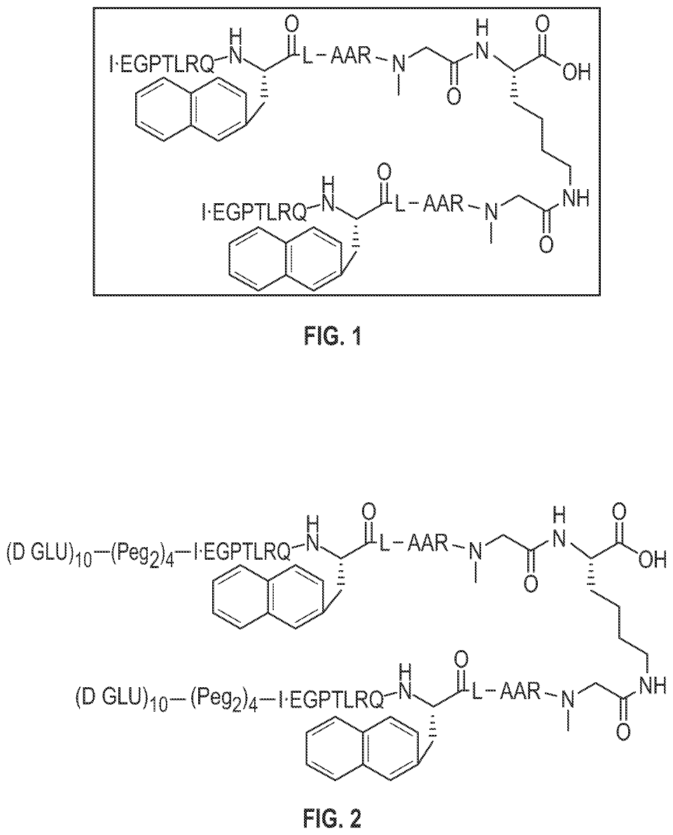

Dimerized thrombopoletin mimetic peptides (TMP) have proven to have essentially the same activity as the full TPO protein. AF13948, the dimer of AF12505, has proven to have particularly high potency and stability and was synthesized to be used as a systemically administered drug. It was demonstrated that the PEGylation of the N-terminus did not result in a reduction of potency of AF13948, To avoid interfering with binding to the CD110 receptor, we attached TMP via a hydrophilic linker of four PEG₂ (amino-ethoxy ethoxy acetic acid (AEEA)) molecules to a chain of 10 D-glutamic acids. A targeted TMP (e.g., as shown in FIG. 2) was synthesized according to standard Fmoc solid-phase peptide synthesis procedures. TMP was synthesized in a solid-phase peptide synthesis vial under a stream of argon. 2-chlorotrityl resin (0.6 mmol/g) was loaded at 0.6 mmol/g with Nα,Nε-di-Fmoc-L-lysine for 60 mins in dichloromethane (DCM) and diisopropylethylamine. The resin was then capped with four washes of HPLC-grade MeOH, followed by three washes with DCM and DMF, consecutively. After each amino acid coupling reaction, Fmoc aroups were removed by two 10-min incubations with 20% (v/v) piperidine in DMF. The resin was then washed twice with DMF prior to adding the next amino acid. Each amino acid was reacted in a three-fold excess with HBTU/NMM for 30 mins, followed by a double coupling with three-fold excess PyBOP/NMM for 30 mins. All amino acids were added according to the conditions above. Standard Fmoc-protected amino acids with acid-sensitive, side chain-protecting groups were used, unless otherwise noted.

Thereafter, the following peptide sequence was added onto the peptide using the solid-phase procedures listed above using the AAPPTec Focus XC automated peptide synthesizer: (D) Glu₁₀(AEEA)₄-lie-Glu-Gly-Pro-Thr-Leu-Arg-Gln-Npa-Leu-Ala-Ala-Arg-Sar. Upon completion of the synthesis, the terminal Fmoc was removed using the same conditions as listed above. Next, the resin was washed three times with DMF, three times with DCM, and two times with methanol, and then dried with argon gas. The dried resin with the peptide was cleaved using 95:2,5:2.5 trifluoroacetic acid/water/tri-isopropyl silane for 2 hrs. The peptide was then precipitated from the cleavage solution using 10 times the volume of cold diethyl ether. The solution was spun at 2,000 rcf for 5 mins and then decanted. The pellet was then desiccated and submitted to LCMS for confirmation of synthesis. The crude peptide was dissolved in a mixture of DMF and water and purified via preparative reversed-phase high-performance liquid chromatography. To purify the TMP, a 0-18 column with a 0-50% ammonium acetate/acetonitrile mobile phase for 40 mins was used. The fraction that contained only pure targeted TMP as assessed by LCMS was lyophilized and stored at −20° C.

All compounds were tested at three different logarithmic doses. All compounds were dissolved in sterile, phosphate-buffered saline at the following concentrations: 1 μM, 10 μM, 100 μM, and 1 nM in order to deliver 100 pmols, 1 nmol, 10 nmol, or 100 nmol per 100 μL injection. These doses were chosen because the expected effective dose for each of the identified compounds fell in this range and compounds with EC₅₀'s in this range can make successful targeted drugs. The formulated drugs were aliquoted into labeled tubes that were frozen and thawed only once for a single-day dose administration.

### Example 2

This example demonstrates the efficacy of a targeted TMP conjugate for healing a fracture in vivo in female Swiss Webster mice with midshaft femur fractures.

Midshaft femur fractures were induced in female Swiss Webster mice (n=5). Aseptic surgical techniques were used to place a 23-gauge needle as an intramedullary nail in the femur of anesthetized, 12-week-old, 28-gram Swiss Webster age-matched mice for internal fixation before fracture. The hair surrounding the right knee of the hind paw was removed from each mouse. The animals were then anesthetized using 3% isoflurane. Their skin was cleaned with a scrub of betadine followed by a scrub of 70% ethanol. An incision was made over the patella, and the patellar tendon was exposed on each mouse. The patellar tendon was transected to expose the distal condyles of the femur. A sterile 23-gauge needle was drilled through the cortical shell of the center of the patellar surface at the distal femur between the condyles. The pin was inserted down the center of the medullary cavity until it reached the endosteal surface of the proximal epiphysis of the femur. The needle was then cut with wire cutters to be flush with the distal end of the femur. The skin was closed with 4-0 non-absorbable nylon sutures.

The femur fractures were induced using a drop-weight fracture device from RlSystem AG, dropping a weight of 200 g from a height of 10 cm. Fractures were verified via X-ray with a Kodak Carestream system. The mice received buprenorphine (0.003 mg/day) for 3 days post-fracture. The mice were dosed subcutaneously with conjugate each day for 3 weeks (some of the following studies were done at 2 weeks and 4 weeks in order to find the optimal window for fracture healing) with an n=5-10 for each of the different drug dose combinations. Two weeks proved to be too short because mechanical studies were difficult due to the instability of the fracture callus at that point. At four weeks, the difference between the control and treated groups decreased as the untreated group began to catch back up. Fracture healing was assessed using micro-CT (Scanco Medical Ag or PerkinElmer). The micro-CT was scanned at high resolution with a voxel size of 10 μM. The PerkinElmer Quantum FX micro-CT or Scanco Medical AG was run at 90 kV and 88 uA, with a 14-minute scan time (PerkinElmer) or 4-hour scan time (Scanco) and an Al 0.5 mm+Cu 0.06 mm filter, The scanning detector rate was 117 fps. The images we analyzed in ImageJ using the BoneJ package. Morphometric parameters were quantified in an ROI that included just the fracture callus. Trabecular thickness (Tb.Th.), trabecular spacing (Tb.Sp.), total volume (TV), and volume of calcified callus (BY) were calculated. Fractured femurs were tested for strength in a four-point bend-to-failure using a Dual Column 5960 (Instron) with a 500 N load cell. Lower supports were 10 mm apart on the anterior face of the femur in contact with the proximal and distal diaphysis. Upper supports were 4 mm apart and spanned the entire fracture callus on the diaphysis. Force was applied from the posterior face of the femur with a displacement rate of 0.3 mm/second. Peak load, yield load, stiffness, post-yield displacement, work-to-fracture, and deformation data were generated. Statistical analysis was performed using a one-way analysis of variance (ANOVA) and a Dunnett's post-hoc analysis with significance reported at the P value of 0.05. All animal experiments were performed in accordance with protocols approved by Purdue University's Institutional Animal Care and Use Committee (IACUC).

Following the diaphysis of broken femurs that contained the fracture calluses, the entire lengths of healthy femurs were scanned using high-resolution microcomputed tomography (μCT 40; SCANCO Medical AG, Brüttisellen, Switzerland) after removal of the stabilization pins from the femurs. Images were acquired at a tube potential of 70 kVp, X-ray intensity of 113 μA and 300 ms integration time, with voxel sizes of 8×8×8 μm³. Slide images (2048×2048) were analyzed with ImageJ (version 1.51) containing BoneJ plugin (version 1.4.2), One hundred continuous slices with the largest diameter were selected from each image to represent the center portion of the fracture callus. Manual contouring of the fracture callus region was performed on the first, last and middle slices, followed by automated interpolation for all remaining slices. 3D image reconstruction of those slices were used to calculate bone density (bone volumeltotal volume, or BV/TV), trabecular thickness (Tb.Th), and trabecular separation (Tb.Sp) according to Bouxsein, Dempster, and their colleagues.

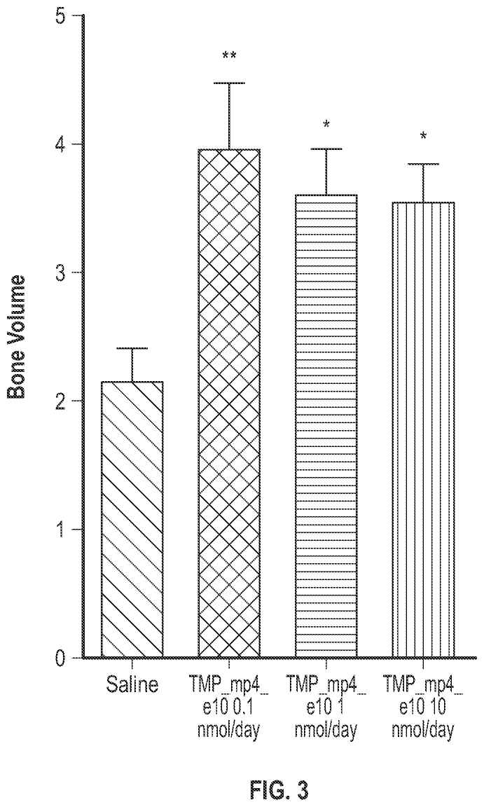

Bone volume was measured for the 100 thickest micro-CT slices of the fracture callus. Bone volume is a measure of how much bone has mineralized at the site of fracture repair. The results are shown in FIG. 3, which is a graph of saline (control) and three different daily doses (0.1, 1 and 10 nmol) of targeted TMP conjugate administered subcutaneously vs. bone volume (100 thickest micro-CT slices of fracture callus) in female Swiss Webster fracture-bearing mice (n=5) after three weeks of treatment. The results show that at higher dose levels, the bodies of the mice started downregulating the response.

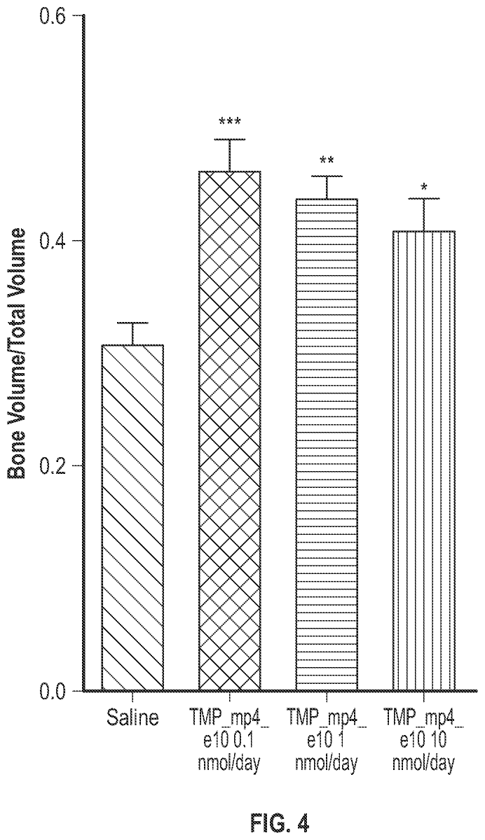

Bone volume/total volume was also measured after three weeks. Bone volume,/total volume represents the bone volume divided by the total volume of the 100 thickest micro-CT slices of the fracture callus. Bone volume/total volume is a measure of how dense the bone is at the site of fracture repair. The results are shown in FIG. 4, which is a graph of saline (control) and three different daily doses (0.1, 1 and 10 nmol) of targeted TMP conjugate administered subcutaneously vs. bone volume/total volume (100 thickest micro-CT slices of fracture callus) in female Swiss Webster fracture-bearing mice (n=5) after three weeks of treatment. The results show that at higher doses, the bodies of the mice started downregulating the response.

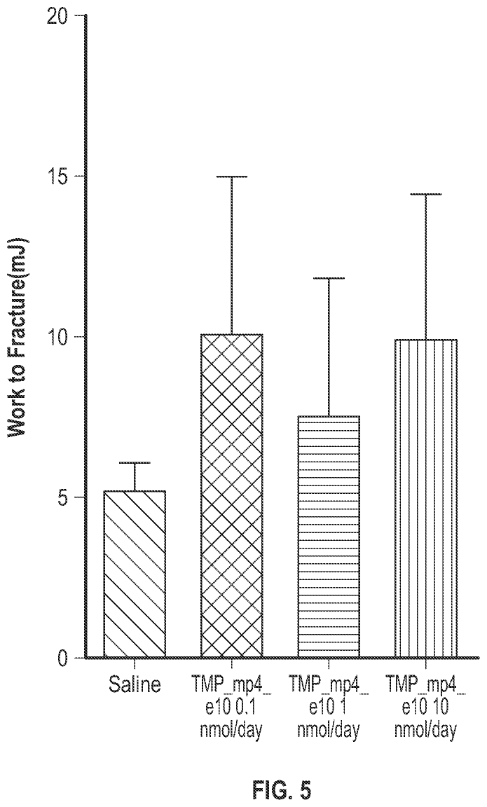

Work to fracture (mJ) was also measured after three weeks. Work to fracture represents the total amount of energy absorbed by the healed femur before it refractured in a postmortem four-point bend analysis. Work to fracture is a measure of how strong the bone is at the site of fracture repair. The results are shown in FIG. 5, which is a graph of saline (control) and three different daily doses (0.1, 1 and 10 nmol) of targeted TMP conjugate administered subcutaneously vs. work to fracture (mJ; total amount of energy absorbed by healed femur before refracture in post-mortem four-point bend analysis) in female Swiss Webster fracture-bearing mice (n=5) after three weeks of treatment. The results show that administration of the targeted TMP conjugate increased the total amount of energy absorbed by the healed femur before it refractured.

### Example 3

This example demonstrates the efficacy of untargeted TMP for healing a fracture in vivo in female Swiss Webster mice with midshaft femur fractures.

Midshaft femur fractures were induced in Swiss Webster mice (n=5) as described above. The mice were treated with untargeted TMP daily for three weeks by subcutaneous injection.

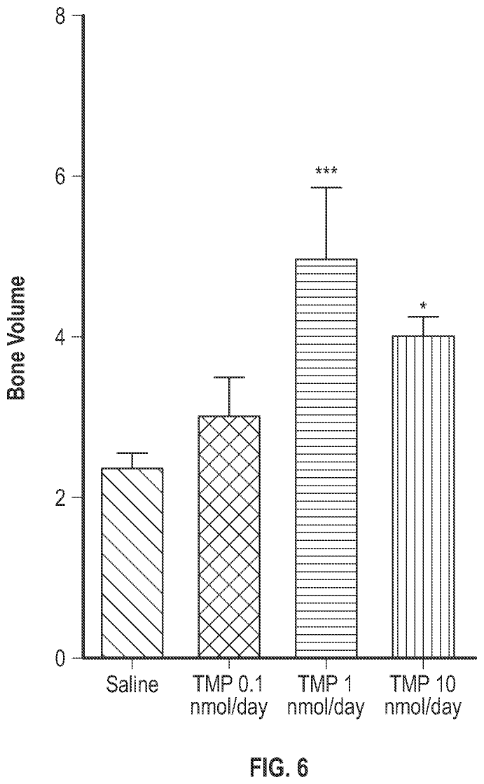

Micro-CT was performed as described above. Bone volume was measured for the 100 thickest micro-CT slices of the fracture callus. Bone volume is a measure of how much bone has mineralized at the site of fracture repair. The results are shown in FIG. 6, which is a graph of saline (control) and three different daily doses (0.1, 1 and 10 nmol) of untargeted TMP administered subcutaneously vs. bone volume (100 thickest micro-CT slices of fracture callus) in female Swiss Webster fracture-bearing mice (n=5) after three weeks of treatment. The results show that TMP has a parabolic dosing window. A dose of 1 nmol/day had the maximum response on callus mineralization.

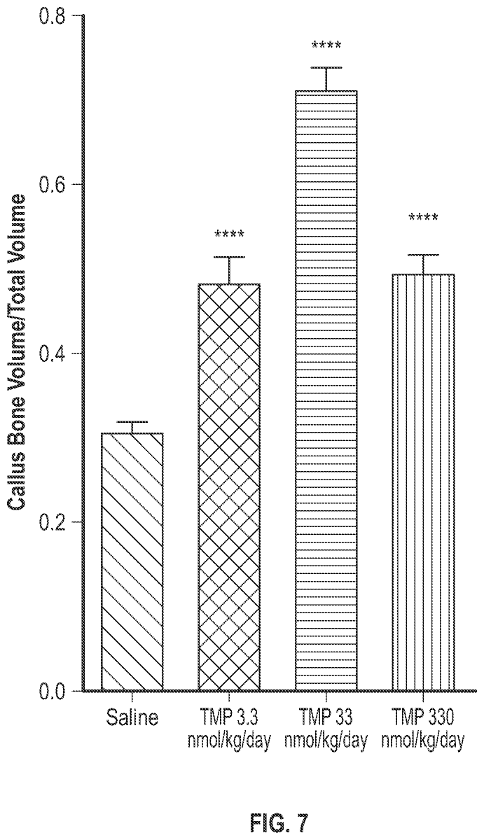

Callus bone volume/total volume was also measured after three weeks. Bone volume/total volume represents the bone volume divided by the total volume of the 100 thickest micro-CT slices of the fracture callus. Bone volumeltotal volume is a measure of how dense the bone is at the site of fracture repair. The results are shown in FIG. 7, which is a graph of saline (control) and three different daily doses (3.3, 33 and 330 nmol/kg) of untargeted TMP administered subcutaneously vs. callus bone volume/total volume in female Swiss Webster fracture-bearing mice (n=5) after three weeks of treatment. The results show that TMP has a parabolic dosing window. A dose of 33 nmol/kg had the maximum response on callus mineralization.

### Example 4

This example demonstrates that lowering the dose of the targeted TMP conjugate improved efficacy.

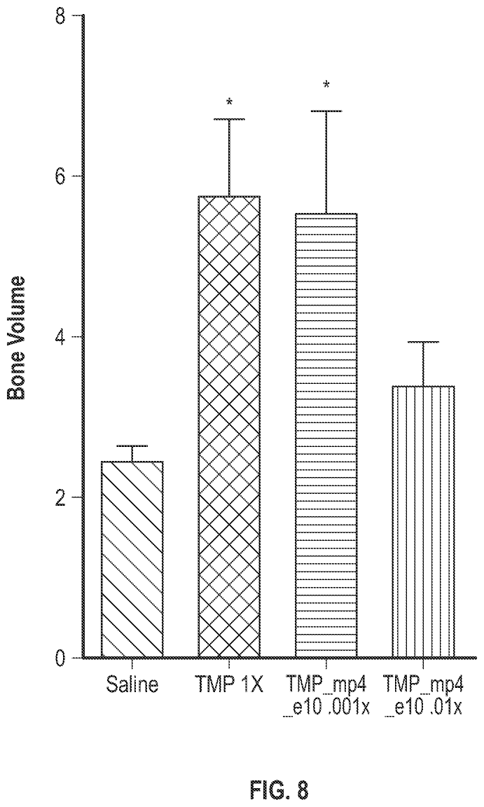

Bone volume was again measured for the 100 thickest micro-CT slices of the fracture callus. The results are shown in FIG. 8, which is a graph of saline (control), 1 nmol/day of TMP, and 0.001 nmol/day and 0.01 nmol/day of targeted TMP conjugate administered subcutaneously vs. bone volume in female Swiss Webster fracture-bearing mice (n=5) after three weeks of treatment. The results show that lowering the dose of the targeted TMP conjugate improved efficacy.

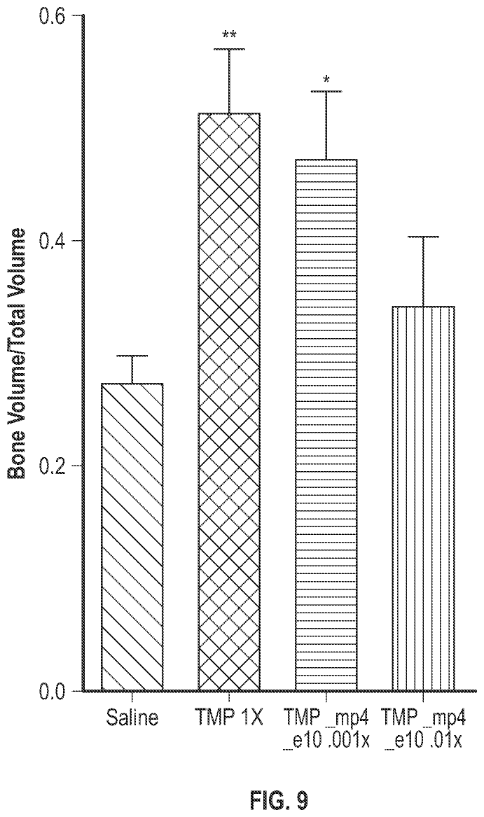

Bone volume/total volume was again measured for the 100 thickest micro-CT slides of the fracture callus, The results are shown in FIG. 9, which is a graph of saline (control), 1 nmol/day of TMP, and 0.001 nmol/day and 0.01 nmol/day of targeted TMP conjugate administered subcutaneously vs. bone volume/total volume in female Swiss Webster fracture-bearing mice (n=5). The results show that lowering the dose of the targeted TMP conjugate improved its efficacy.

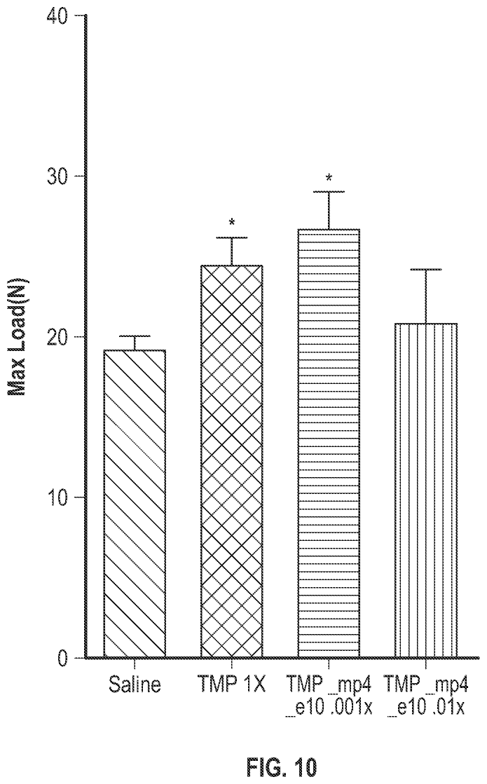

Maximum load was also measured after three weeks. Maximum load represents the maximum force the healed femur withstood before it refractured in a post-mortem four-point bend analysis. FIG. 10 is a graph of saline (control), one dose of TMP, and two doses of targeted TMP conjugate administered vs. maximum (Max) load (N). The results show that lowering the dose of the targeted TMP conjugate improved its efficacy.

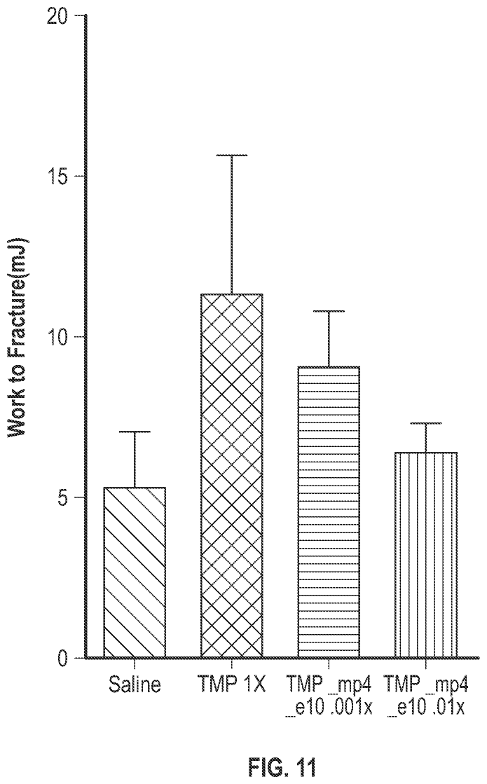

Work to fracture (mJ) was also measured after three weeks. Work to fracture represents the total amount of energy absorbed by the healed femur before it refractured in a postmortem four-point bend analysis. Work to fracture is a measure of how strong the bone is at the site of fracture repair. The results are shown in FIG. 11, which is a graph of saline, 1 nmol/day of TMP, and 0.001 nmol/day and 0.01 nmol,/day of targeted TMP conjugate administered subcutaneously vs. work to fracture (mJ) in female Swiss Webster fracture-bearing mice (n=5), The results show that lowering the dose of the targeted TMP conjugate improved its efficacy.

### Example 5

This example demonstrates that reducing the frequency of dosing maximizes the therapeutic efficacy of targeted TMP conjugate.

Midshaft femur fractures were induced in Swiss Webster mice (n=5) as described above. The mice were treated with TMP or targeted TMP conjugate by subcutaneous injection every three days for three weeks or every day for seven days.

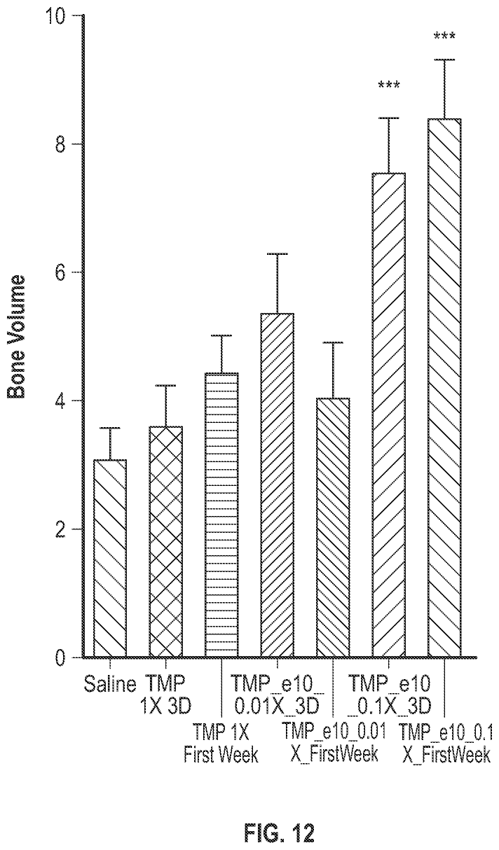

Bone volume was measured for the 100 thickest micro-CT slices of the fracture callus, Bone volume is a measure of how much bone has mineralized at the site of fracture repair. The results are shown in FIG. 12, which is a graph of saline, 1 nmol/day of TMP administered subcutaneously either every three days for three weeks or once a day for one week, either 0.01 nmol/day or 0.1 nmol/day of targeted TMP conjugate administered subcutaneously every three days for three weeks, and either 0.01 nmol/day or 0.1 nmol/day of targeted TMP conjugate administered subcutaneously once a day for one week vs, bone volume in female Swiss Webster fracture-bearing mice (n=5). The results show that at higher dose levels, the bodies of the mice started downregulating the response, The results show that the therapeutic effect is maximized by reducing the number of doses and primarily affects the inflammatory phase of fracture healing.

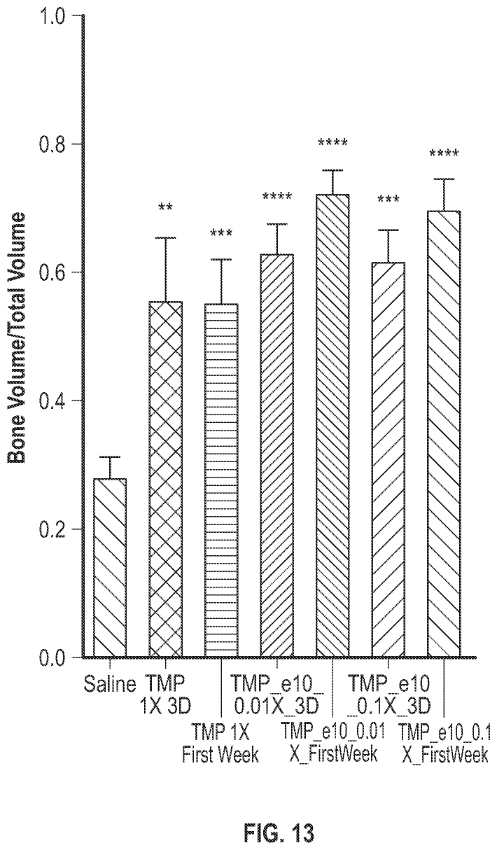

Bone volume/total volume was also measured after three weeks. Bone volume/total volume represents the bone volume divided by the total volume of the 100 thickest micro-CT slices of the fracture callus. Bone volume/total volume is a measure of how dense the bone is at the site of fracture repair. The results are shown in FIG. 13, which is a graph of saline, 1 nmol/day of TMP administered subcutaneously either every three days for three weeks or once a day for one week, either 0.01 nmol/day or 0.1 nmol/day of targeted TMP conjugate administered subcutaneously every three days for three weeks, and either 0.01 nmol/day or 0.1 nmol/day of targeted TMP conjugate administered subcutaneously once a day for one week vs. bone volume/total volume in female Swiss Webster fracture-bearing mice (n=5). The results show that the therapeutic effect is maximized by reducing the number of doses and primarily affects the inflammatory phase of fracture healing. A daily dose of 0.1 nmol targeted TMP performed the best and exceeded the performance of a daily dose of 1 nmol of untargeted TMP.

### Synthesis of TMP-Deca Oligopeptides

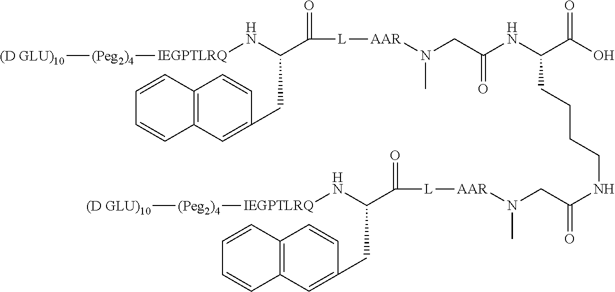

Dimerized thrombopoietin mimetic peptides (TMP) have essentially the same activity as the full TPO protein, and AF13948, the dimer of AF12505, has high potency, The targeted TMP-aspartic acid conjugate was synthesized according to standard Fmoc solid phase peptide synthesis procedures. Briefly, deca-aspartic acid was synthesized in a solid phase peptide synthesis vial under a stream of nitrogen. 2-chlorotrityl resin (0.6 mmol/g) was loaded at 0.6 mmol/g with the first amino acid overnight in DCM and DIPEA. The resin was then be capped with four 5-mL washes of DCM/MeOH/DIPEA (17:2:1), followed by three washes with DCM and DMF, consecutively. After each amino acid coupling reaction, Fmoc groups were removed by three 10-min incubations with 20% (v/v) piperidine in DMF. The resin was then washed 3× with DMF before addition of the next amino acid. Each amino acid is reacted in a 3-fold excess with HBTU/NMM. Fmoc PEG4 carboxylic acid (AAPPTec) was then added to the N-terminus of the deca-aspartic acid to serve as a spacer. A β-alanine-modified lysine with an Fmoc protecting group on its side chain was then added. Thereafter, the following peptide sequence was added onto the peptide using the solid phase procedures: Ile-Glu-Gly-Pro-Thr-Leu-Arg-Gln-Trp-Leu-Ala-Ala-Arg-Ala. Upon completion of the synthesis, the peptide was cleaved using 95:2.5:2.5 trifluoroacetic acid:water:triisopropylsilane. It is expected that the resulting compound will have an ability to stimulate megakaryocyte proliferation and differentiation in vitro.

### Example 6

### Synthesis of SRT1720-Aspartio Acid Oligopeptides

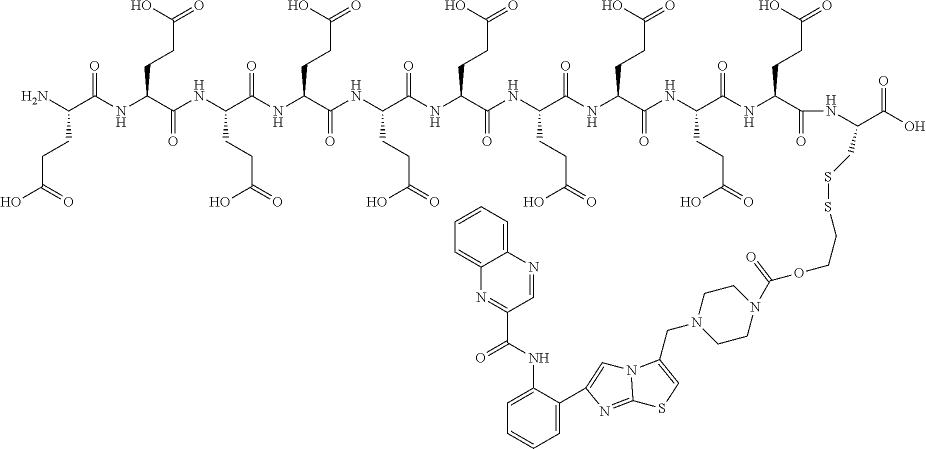 [SMILES: N[C@@H](CCC(=O)O)C(=O)N[C@@H](CCC(=O)O)C(=O)N[C@@H](CCC(=O)O)C(=O)N[C@@H](CCC(=O)O)C(=O)N[C@@H](CCC(=O)O)C(=O)N[C@@H](CCC(=O)O)C(=O)N[C@@H](CCC(=O)O)C(=O)N[C@@H](CCC(=O)O)C(=O)N[C@@H](CCC(=O)O)C(=O)N[C@@H](CCC(=O)O)C(=O)N[C@@H](CSSCCOC(=O)N1CCN(Cc2csc3nc(-c4ccccc4NC(=O)c4cnc5ccccc5n4)cn23)CC1)C(=O)O]

Deca-aspartic add was synthesized using the solid phase peptide synthesis as described in the previous example. Carboxylic acid-PEG4 -alcohol purchased from AAPPTec (Kentucky, USA) was coupled to the N-terminus of the deca-aspartic add with 3-fold excess PyBOP/DIPEA. The resulting acidic peptide was then reacted with triphosgene to form a chloroformate. SRT1720 (purchased from AK Scientific, Inc.; Union City, CA, USA) was then reacted with the above chloroformate deca-aspartic add to form a carbamate between the PEG linker and the secondary amine on SRT1720. This created a cleavable linker that permits release of unmodified SRT1720 upon localization of the full-length conjugate at the fracture site. It is expected that the resulting compound will have an ability to activate Sirt1 expression in osteoblast lineage cells,

Systemic Drug Treatment. The targeted TMP (TMP-Deca Oligopeptide described herein; 3.1, 31, or 310 nmol/kg), targeted SRT1720 (SRT1720-Aspartic Add Oligopeptide described herein; 3.1, 31, or 310 nmol/kg), or vehicle PBS was injected subcutaneously daily.
Bone Union. X-rays will be taken during surgery and each week post-surgery (orthogonal views). Medial (m), lateral (l), anterior (a), and posterior (p) cortices visible on anteroposterior and lateral X-rays will be scored by methods known in the art. Percentage of bone bridging was measured from the images by taking the bone bridging over the synthetic scaffold (each cortice) along with the total length of the scaffold. Additionally, bone union was measured using the Radiographic Union Score for Tibial Fractures (RUST) method (but adapted and applied to the femur) as a semi-quantitative method for analysis. Here the medial, lateral, anterior, and posterior cortices visible on anteroposterior and lateral X-rays were scored. Specifically, each cortex is given a score of 1 (no bridging), 2 (partial bridging), or 3 (complete bridging) and the scores for all 4 cortices are added up to provide a final score ranging from 4 (not healed) to 12 (maximally healed), Bone bridging was independently assessed by three different investigators blinded to treatment group.
μCT Assessments (% bone bridging, callus volume, % mineralized callus volume). The excised femurs sequestered for histology and biomechanical analyses also underwent μCT analysis. Femurs were imaged via μCT (Skyscan 1172) to evaluate the three-dimensional, spatial distribution of the region containing the scaffold/CSD. μCT images were also used to obtain geometry including outer diameter of the callus area as well as polar moment of inertia, to characterize not only the amount of tissue, but its distribution about the central axis. A 3D analysis was performed to measure the following morphometric parameters, in accordance to the American Society for Bone and Mineral Research: (1) TV, in mm³, the total callus volume; (2) BV/TV, in %: the woven bone fraction, which was expressed as a percentage of the callus volume; (3) Conn.D., in 1/mm³: the connectivity density normalized by TV; (4) SMI: the structure model index, where 0 stands for parallel plates and 3 for cylindrical rods; (5) TMD, in 1/cm: mean callus density; (6) BS, in mm²: bone surface area; (7) BS/BV, in 1/mm: relative bone surface; (8) Tb.N, in 1/mm: trabecular number; (9) Tb.Th, in mm: trabecular thickness; and (10) Tb.Sp, in mm: trabecular separation. All surgical femurs (and contralateral femurs) underwent this assessment.

### Torsional Biomechanical Testing (Load at Failure, Stiffness).

For femora undergoing biomechanical analyses, fixed femora were rehydrated in DPBS with calcium and magnesium at room temperature for 16-24 hours. Femora underwent immediate biomechanical torsion analysis after μCT to prevent repeated freeze-thaw cycles and to maintain structural integrity of the bone. Femora were potted into .38 bullet casings and held in place with orthodontic resin (Dentsply Sirona, York, PA). The resin was allowed to harden for an additional 5 minutes prior to testing. Biomechanical testing was then performed in torsion at 1 degree/second until failure using a Mark-10 (Copiague, BY) advanced torque-testing system. Ultimate torque, twist to failure, secant stiffness, manual stiffness, maximal stiffness, toughness, 25% displacement twist to failure, and 25% displacement toughness was calculated from the resultant torque-twist curves. Ultimate torque was defined as the maximum torque sustained, twist to failure was defined as the angular displacement when ultimate torque was obtained, secant stiffness was determined as the slope of the line between the starting point and point of ultimate torque within the torque-twist curve, manual stiffness was defined as the slope of the line between the starting point and point of ultimate torque within the range of 50-75% of the ultimate torque, maximal stiffness was defined as the largest slope of the starting point and point of ultimate torque within a 5 degree angular displacement range, toughness was calculated as the area under the torque-twist curve, 25% displacement twist to failure was defined as the twist to failure after 25% of the ultimate torque was reached within the torque-twist curve, and 25% displacement toughness was defined as the toughness after 25% of the ultimate torque was reached within the torque-twist curve. All properties were normalized to contralateral bones. The 25% displacement twist to failure and 25% displacement toughness was utilized to nullify the initial angular displacement before significant levels of torque will be achieved.

Histological Assessments: Bone Defect Site and Bone Parameters (% cartilaginous callus volume; % intramembranous bone; % endochondral bone; % reticulin fibers; numbers of osteoblasts and osteoclasts in the bone defect site). Femurs were fixed in 10% neutral buffered formalin (NBF) for at least 43 hours and then transferred to 70% EtOH. After μCT scanning, femurs from mice sequestered for histology were demineralized, embedded in paraffin, sectioned longitudinally, and stained with alcian blue/picrosirius red and were evaluated under light microscopy in conjunction with an automated histomorphometry system (Leica DMI6000 with Image Pro analysis software) to obtain callus size, calcified cartilage volume, bone volume in the healing callus, and other relevant bone parameters.

X-ray imaging. X-ray imaging is the most clinically used diagnostic tool to evaluate fracture healing. After surgery, mice received biweekly X-ray imaging using a Kubtec Xpert80 X-ray system. Anteroposterior (AP) and lateral X-rays of mice were taken at 7, 10, 14, 17, 21, and 28 days post-surgery for surgically induced and Eichorn fracture models. Xrays were typically taken weekly or every other week for larger segmental bone defect or critical sized defect injuries.
RUST and mRUST scoring of fracture healing. Fracture healing was assessed using the Radiographic Union Score for Tibial fractures method (RUST). Using this method, the medial, lateral, anterior, and posterior cortices on anteroposterior and lateral μCT or x-ray images were scored. Each cortex was given a score of 1 (no bridging), 2 (partial bridging), or 3 (complete bridging). The total score was equal to the sum of the scores from all 4 cortices ranging from 4 (not healed) to 12 (maximally healed). All images were scored by 3 blinded readers and the average score reported for each specimen.

Bone healing in each X-ray image or uCT image was evaluated by three orthopaedic surgeons using mRUST scoring. The mRUST assigns integer scores to each cortex imaged on the anteroposterior (medial and lateral cortices) and lateral (anterior and posterior cortices) X-rays as follows: 1 no healing; 2 callus present, no bridging; 3 bridging callus, fracture line visible; 4 bridging callus with no fracture line visible. The scores for all 4 cortices were added up to provide a final score ranging from 4 (not healed) to 16 (maximally healed). Mean mRUST scores were calculated from all observations made by the three surgeons for each specimen at each time point. Also determined were week-to-week changes in mRUST scores in each group (Delta mRUST). In addition, individual and mean mRUST scores were ≥11 and ≥13, as these scores that are likely reflective of sufficient healing.

### Example 7

Non-union bone fracture occurs in 5-10% of fracture injuries. Interventions include surgery with local implantation of autograft, allograft, demineralized bone matrix, and/or bone morphogenetic proteins.

Fracture injuries are accompanied by acute and chronic pain states. Opioids after chronic exposure are known to cause opioid-induced hyperalgesia, thus reducing mechanical loading which is critical for bone healing. With the opioid crisis, identifying new analgesic therapies that could reduce or eliminate opioid use, while also improving bone healing is critical.

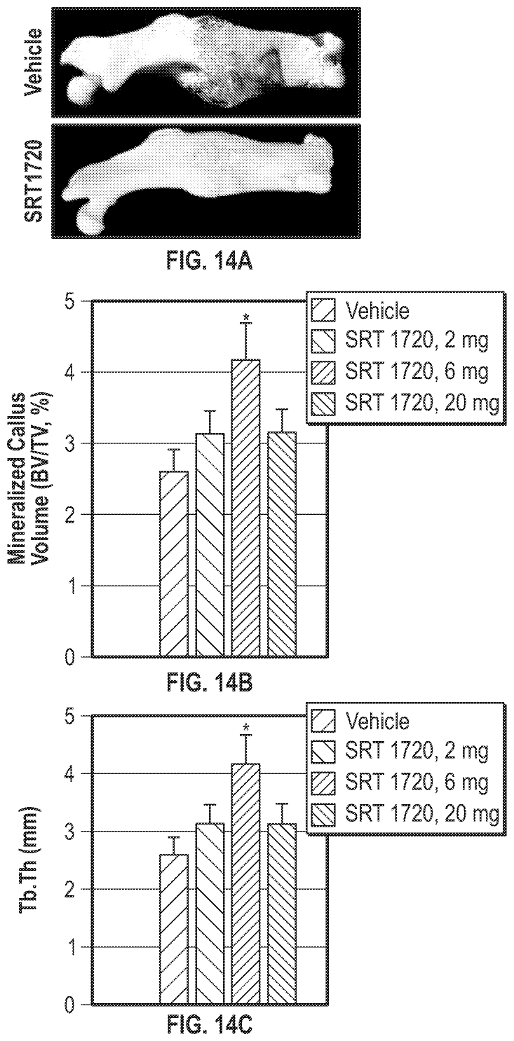

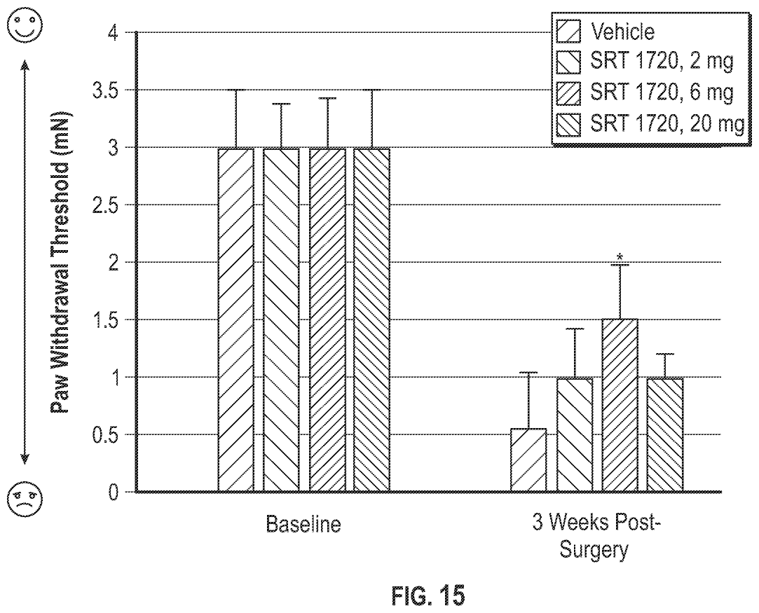

The compound SRT1720 targets a protein upregulated during bone healing. Studies have shown SRT1720, to both enhance bone healing and reduce pain behavior in a surgically induced femoral fracture mouse model. Briefly, 20 male C57B1.16 mice underwent a surgically induced femoral fracture and then were treated with 0, 2, 6, or 20 mg/kg, 3×/week for the 3-week study duration. Weekly X-rays were used to examine healing progression. Prior to euthanasia, mice underwent behavioral testing to measure evoked pain behaviors, Upon euthanasia, ex vivo μCT imaging (FIGS. 14A-14C) and analysis was completed to assess fracture callus size and composition. While all doses of SRT1720 tested resulted in improved healing, the 6 mg/kg dose resulted in accelerated bone healing and a significant increase in mineralized callus volume (p<0.05). Similarly, while all doses of SRT1720 reduced evoked responses to tactile stimulus as demonstrated by increased paw withdrawal thresholds, 6 mg/kg of SRT1720 resulted in a more robust and significant improvement (p<0.05) (FIG. 15).

### Example 8

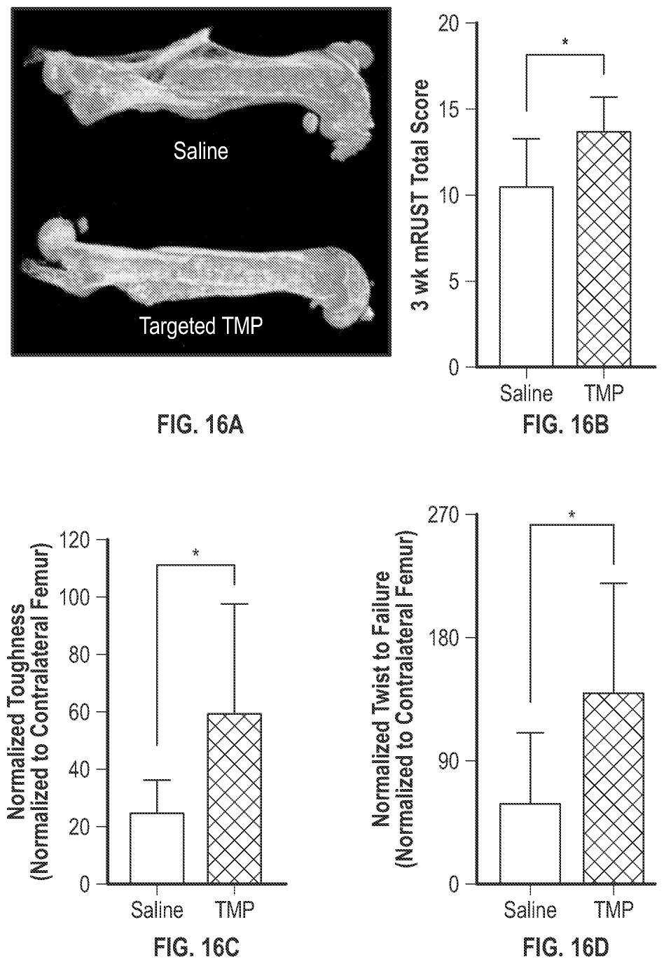

Studies using surgically created fracture model in 12-week-old, male C57BL/6 mice were completed. μCT images of fractured femurs were collected from mice treated daily for the first week post-surgery with saline or targeted TMP (also referred to herein as “TMP e10” and “TMP-deco oligopeptides) (FIG. 16A) and examined 3 weeks later. TMP-treated femurs exhibit complete bone bridging/union and improved mineralization and bone remodeling compared to saline controls. Modified radiographic union score for tibia fractures (mRUST) analyses show that TMP-treated mice exhibit complete bridging (bone union) on all cortices, but only 1 of 6 saline treated mice showed complete bridging (FIG. 16B). Also observed were improved biomechanical torsion properties, toughness and twist to failure, respectively, of injured femurs normalized to their contralateral femurs. Together, these findings suggest that targeted TMP administered only during the first week post-injury improves fracture healing (FIGS. 16C and 16D).

### Example 9

The prevalence of chronic neuropathic pain following orthopedic surgeries is about 1 in 10 for adults over 30 years of age. This Example assesses how fracture-targeted TMP alone impacts pain states in fracture heating including weightbearing analyses as one measure of pain behaviors.

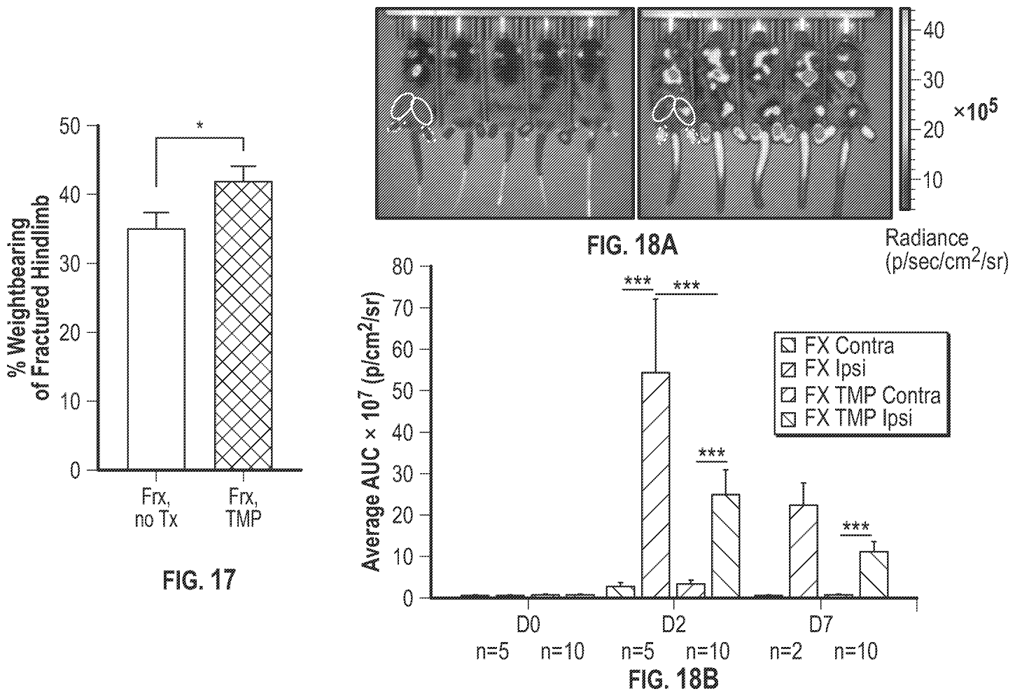

Weight bearing between rear hindlimbs was evaluated in uninjured mice and found to be approximately 50-50 (52.1:±1.4% in right vs. left limbs). Weightbearing was then assessed, 4 days post-surgery in mice with a surgically created fracture in their right femur. Mice treated with targeted TMP (3.3 nmol/kg/day) exhibited significantly higher weightbearing on their injured leg (42.0±2.1%) than did injured controls (35.3±2.0%) (p=0.04, n=7/group) (FIG. 17), This suggests that taraeted TMP can improve pain-associated functional outcomes in mice in addition to bone healing.

### Example 10

As excessive inflammation and poor resolution of inflammation are central to pathogenesis of impaired fracture healing and development of chronic pain, we also assessed local inflammation following targeted TMP treatment in live animals. Specifically, luciferase imaging of caspase-1 biosensor mice (C57BL/6 transgenic mice constitutively expressing the caspase-1 biosensor) was carried out to examine the extent and location of inflammation during bone healing. In these mice, a known caspase-1 target sequence was introduced into a circularly permuted luciferase construct that becomes bioluminescent upon protease cleavage to monitor caspase-1 activation in vivo. There was virtually no signal at baseline (D0) in young, healthy, uninjured mice (FIG. 184). However, 2 days after fracture surgery, caspase-1 was markedly upregulated in the injured limb and remained high 1-week post-surgery (FIG. 18B). Limited signal was observed in contralateral limbs at any time. Interestingly, mice provided with targeted TMP for the first week post-surgery (3.3 nmol/kg/day), exhibited a marked reduction in caspase-1 activation compared to saline controls (FIG. 18B).

### Definitions

“Angiogenic” refers to the ability to induce migration, proliferation and/or differentiation of endothelial cells leading to new blood vessel formation.

“Bone” generally refers to a mineralized tissue primarily comprising a composite of deposited calcium and phosphate in the form of hydroxyapatite, collagen (primarily Type I collagen) and bone cells such as osteoblasts, osteocytes and osteoclasts, as well as to bone marrow tissue. Bone is a vascularized tissue.

Bone is generally in the form of “compact bone” (or “cortical bone”) or “spongy bone” (or “cancellous bone” or “trabecular bone”). From a gross anatomical perspective there are clear differences between compact and spongy bone. Specifically, compact bone has a lamellar structure and generally represents a dense area of bone tissue that does not contain cavities, whereas spongy bone contains numerous interconnecting cavities defined by complex trabeculae, Compact bone is typically harder, stronger and stiffer than cancellous bone. The higher surface area to mass ratio of cancellous bone compared to compact bone means that cancellous bone is less dense than compact bone and is generally softer, weaker and more compliant than compact bone. Cancellous bone is highly vascularized and is typically found at the ends of long bones, proximal to joints and within the interior of vertebrae. Compact bone typically forms a “shell” around cancellous bone and is the primary component of the long bones of the arm and leg and other bones, where its greater strength and rigidity are needed. The primary anatomical and functional unit of compact bone is the osteon and the primary anatomical unit of cancellous bone is the trabecula.

“Bone anabolic compound” refers to compounds that stimulate new bone formation but do not necessarily have catabolic, angiogenic, and/or thrombopoietic properties. Parathyroid hormone (PTH), PTH related peptide (PTHrP), and bone morphogenetic protein (BMP) are examples of bone anabolic compounds.

“Bone defect” refers to a structural disruption of bone requiring repair, A bone defect can assume the configuration of a “fracture” which means a break such, as for example a complete fracture or a partial fracture, or a “void,” which means a three-dimensional defect such as, for example, a gap, cavity, hole or other substantial disruption in the structural integrity of a bone or joint. A defect can be the result of accident, disease, surgical manipulation, and/or prosthetic failure. The defect may be a void having a volume incapable of endogenous or spontaneous repair. Generally, these are capable of some spontaneous repair, albeit biomechanically inferior. A “nonunion” is a defect that has not healed in 3 months. A “critical sized defect” is a bone defect that will not heal without intervention. A “delayed healing fracture” is a bone fracture that does not heal or is not expected to heal within a normal time period. Thus, for example, “bone defect” includes bone defects resulting from bone resorption, bone trauma, osteolytic lesions (e.g., osteolytic lesions resulting from osteoporosis, cancers, and bone disease), fractures, delayed unions, and the like.

“Bone bridging” refers to the amount of fracture gap that is bridged by callus.

“Bone marrow microenvironment” refers to a cellular compartment and a non-cellular compartment. The cellular compartment can be subdivided into hematopoietic cell types including myeloid cells, T lymphocytes, B lymphocytes, natural killer cells, and osteoclasts, while non-hematopoietic cells include bone marrow stromal cells (BMSCs), fibroblasts, osteoblasts, endothelial cells, and blood vessels. The non-cellular compartment includes the extracellular matrix (ECM), oxygen concentration, and the liquid milieu (cytokines, growth factors, and chemokines), which are produced and/or affected by the cellular compartment within the bone marrow microenvironment.

“Co-administration” or “co-administering” refers to administration of a disclosed compound with at least one other therapeutic agent within the same general time period, and does not require administration at the same exact moment in time (although co-administration is inclusive of administering at the same exact moment in time). Thus, co-administration may be on the same day or on different days, or in the same week or in different weeks. The additional therapeutic agent may be included in the same composition as the disclosed compounds.

“Dimer” as applied to peptides refers to molecules having two peptide chains associated covalently or non-covalently, with or without linkers. Peptide dimers wherein the peptides are linked C-terminus to N-terminus may also be referred to as “tandem repeats” or “tandem dimers.” Peptide dimers wherein the peptides are linked C- to C-terminus, or N- to N-terminus may also be referred to as “parallel repeats” or “parallel dimers.”

“Exogenous” refers to molecules originating from an outside source with respect to the recipient or otherwise treated subject, An exogenous thrombopoietin mimetic is not naturally occurring or naturally present in the recipient subject.

“Hydrolysable” linker refers to a linker system, in which the molecule having thrombopoietic activity and/or the sirtuin activator is released in native form. The molecule having thrombopoietic activity and/or sirtuin activator is released and the linker is split off partially or completely. Synonyms for hydrolyzable are “degradable” or “releasable” linkers. The hydrolyzable linker provides a means of attaching the molecule having thrombopoietic activity and/or the sirtuin activator to the inventive compounds and the presence of the hydrolyzable group allows the bone healing activity molecules to be detached from the inventive compounds.

“Long bones” are generally bones in which compact bone is found at the diaphysis, which is the cylindrical part of the bone, whereas the spongy bone is found at the epiphyses, i.e. the bulbous ends of a bone. Long bones provide strength, structure, and mobility. A long bone has a shaft and two ends. Long bones typically are longer than they are wide. Examples of long bones include the femur, fibula, humerus, radius, tibia and ulna.

“Bone healing” refers to the processes associated with returning structural continuity to a bone defect. Secondary healing includes four steps: 1, hematoma formation; 2. fibrocartilaginous callus formation; 3. bony callus formation; and, 4. bone remodeling, Primary bone healing is the reestablishment of the cortex without the formation of a callus.

“Bone defect” refers to any bone loss.

“Bone-targeting molecule” refers to any chemical compound that has an affinity for bone, exposed bone mineral, matrix and/or cells, including bone hydroxyapatite, osteocytes, osteoblasts, osteoclasts or any combination thereof on an unexposed or exposed surface of a bone and is capable of selectively targeting bone, bone mineral, matrix and/or cells including hydroxyapatite, osteocytes, osteoblasts, osteoclasts, or any combination thereof over other cells and tissues. Bone-targeting molecules are known to those skilled in the art. See, for example, U.S. Pat. Nos. 8,772,235, 8,703,114, U.S. Patent Application Publication No. 2019/0275160, U.S. Pat. No. 10,744,203, U.S. Patent Application Publication No. 2014/0056855, and WO 1992/20371.

“Mammals” refers to humans, livestock animals (e.g., cows, sheep, goats, pigs), companion animals (e.g., cats and dogs), laboratory animals (e.g., primates, rats, mice, rabbits), and domesticated quadrupeds such as horses.

“Oligopeptide” refers to two or more linked amino acids. Typically, an oligopeptide has two to fifty, more typically four to forty linked amino acids. In embodiments of the invention, oligopeptides include from 4 to 30, or not less than 4 and not more than 20 amino acids, or not less than 4 and not more than 15 amino acids, or not less than 4 and not more than 10 amino acids, or less than 4 and not more than 8; the number of amino acids in the oligonucleotide may be 1, 2, 3, 4, 5, 6, 7, 8, 9, 10, 11, 12, 13, 14, 15, 16 17, 18, 19, 20, or greater. Oligopeptide useful in the present invention have a net negative charge and/or are acidic.

“Amino acid” includes naturally occurring amino acids as well as synthetic amino acids.

“Pharmaceutically acceptable carrier” refers to any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

A “solvate” is formed by the interaction of a solvent and a compound. Solvates of salts of the compounds provided are also provided. Hydrates of the compounds are also provided.

A “prodrug” is a biologically inactive derivative of a drug that upon administration to the human body is converted to the biologically active parent drug according to some chemical or enzymatic pathway.

The term “pharmaceutically acceptable salt” of a given compound refers to salts that retain the biological effectiveness and properties of the given compound, and which are not biologically or otherwise undesirable. Pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, mono, di or tri cycloalkyl amines, mono, di or tri arylamines or mixed amines, etc. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like,

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic adds include acetic add, propionic add, glycolic add, pyruvic add, oxalic add, malic add, malonic add, succinic add, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

“Pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated.

“Small molecule” refers to a low molecular weight organic compounds that may help regulate a biological process. Small molecules include any molecules with a molecular weight of about 2000 daltons or less, preferably of about 500 to about 900 daltons or less. These compounds can be natural or artificial. Biopolymers such as nucleic acids and proteins, and polysaccharides (such as starch or cellulose) are not small molecules—though their constituent monomers, ribo- or deoxyribonucleotides, amino acids, and monosaccharides, respectively, are often considered small molecules. Small molecules include pharmaceutically acceptable salts of small molecules.

“Subject” refers to any refers to either a human or a non-human patient for whom diagnosis, treatment, or therapy is desired. Subjects can be mammal or non-mammal (vertebrate). Examples of subjects include, but are not limited to, humans, livestock (e.g., horses, cows, and pigs), chickens, fish, and the like.

“Therapeutically effective amount” is that amount sufficient, at dosages and for periods of time necessary, to achieve a desired, safe therapeutic result, such as for treatment of a long bone segmental defect, and/or pharmacokinetic or pharmacodynamic effect of the treatment in a subject. A therapeutically effective amount can be administered in one or more administrations. The therapeutically effective amount may vary according to factors such as the disease state, age, sex, and weight of the subject. One skilled in the art will recognize that the condition of the individual can be monitored throughout the course of therapy and that the effective amount of a compound or composition that is administered can be adjusted accordingly.

“Treat” or “treating” is an approach for obtaining beneficial or desired results including clinical results. Beneficial or desired clinical results may include one or more of the following: a) decreasing one or more symptoms resulting from the bone defect, and/or diminishing the extent of the bone defect or condition); b) slowing or arresting the development of one or more clinical symptoms associated with the bone defect (e.g., stabilizing the defect or condition, and/or preventing or delaying the worsening or progression of the defect or condition; and/or c) relieving the defect, that is, causing the regression of clinical symptoms (e.g., ameliorating the defect, providing partial or correction of the defect or condition, healing the defect, repairing the defect or regenerating the defect, enhancing effect of another medication, increasing the quality of life, and/or prolonging survival).

The terms and expressions, which have been employed, are used as terms of description and not of limitation. There is no intention in the use of such terms and expressions of excluding any equivalents of the features shown and described or portions thereof.

It is recognized that various modifications are possible within the scope of the claimed invention. Thus, it should be understood that, although the present invention has been specifically disclosed in the context of preferred embodiments and optional features, those skilled in the art may resort to modifications and variations of the concepts disclosed herein. Such modifications and variations are considered to be within the scope of the invention as claimed herein.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z, ” unless indicated otherwise.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading can occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the steps can be carried out in any order without departing from the principles of the invention, except when a temporal or operational sequence is explicitly recited. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The term “about” as used herein can allow fora degree of variability in a value or range, for example, within 10%, within 5%, or within 1% of a stated value or of a stated limit of a range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

As used in the present specification, the following words, phrases and symbols are generally intended to have the meanings as set forth below, except to the extent that the context in which they are used indicates otherwise.

A dash (“-”) between two letters indicates an attachment or linkage. Unless chemically or structurally required, no specific points of attachment or directionality is indicated or implied by a dash or the order that a chemical group is written or named.

Certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the chemical groups represented by the variables are specifically embraced by the present disciosure and are disclosed just as if each and every combination was individually and explicitly disclosed, to the extent that such combinations embrace compounds that are stable compounds (i.e., compounds that can be isolated, characterized, and tested for biological activity). In addition, all sub-combinations of the chemical groups listed in the embodiments describing such variables are also specifically embraced by the present disclosure and are disclosed just as if each and every such sub-combination of chemical groups was individually and explicitly disclosed.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

1. A conjugate of the formulae I-V:
X—Y¹-Z (formula I);
X-Z (formula II);
X—X—Y¹-Z (formula III);
X—X-Z (formula IV); or
Z-Y¹-Z (formula V)
or a pharmaceutically acceptable salt thereof, wherein:
X is a radical of a molecule having thrombopoietic activity or sirtuin activity and X—X is a dimer of a radical of a molecule having thrombopoietic activity or sirtuin activity;
Y¹ , when present, is a releasable or non-releasable linker, and
Z is an osteotropic ligand.

2. The conjugate of claim 1, or a pharmaceutically acceptable salt thereof, wherein the compound of the formula I is a conjugate of the formula Z-(releasable linker)-X, TMP-(releasable linker)-X or AOP-(releasable linker)-X, wherein TMP represents a thrombopoietin mimetic peptide and AOP represents an acidic oligopeptide.

3. The conjugate of claim 2, or a pharmaceutically acceptable salt thereof, wherein the TMP is AF12505 (IEGPTLRQWLAARA), a modified dimer of AF12505, AF13948, AF13948 in which tryptophan has been substituted with naphthalene and alanine has been substituted with sarcosine, or GW395058 (dimer of AF12505).

4. The conjugate of claim 2, or a pharmaceutically acceptable salt thereof, wherein the osteotropic ligand is an AOP.

5. The conjugate of claim 2, or a pharmaceutically acceptable salt thereof, wherein the AOP comprises at least 6 amino acids up to, and including, 20 amino acids.

6. The conjugate of claim 2, or a pharmaceutically acceptable salt thereof, wherein the AOP comprises aspartic acid, glutamic acid, aminoadipic acid, or a combination of two or more of the foregoing.

7. The conjugate of claim 2, or a pharmaceutically acceptable salt thereof, wherein the AOP comprises amino acids having D chirality, L chirality, or a mixture thereof.

8. The conjugate of claim 2, or a pharmaceutically acceptable salt thereof, wherein the AOP is linear.

9. The conjugate of claim 2, or a pharmaceutically acceptable salt thereof, wherein the AOP comprises one or more neutral or basic amino acids.

10. The conjugate of claim 2, or a pharmaceutically acceptable salt thereof, wherein the AOP comprises one or more non-natural amino acids.

11. The conjugate of claim 1, or a pharmaceutically acceptable salt thereof, wherein the compound of the formula I is a conjugate of the formula: 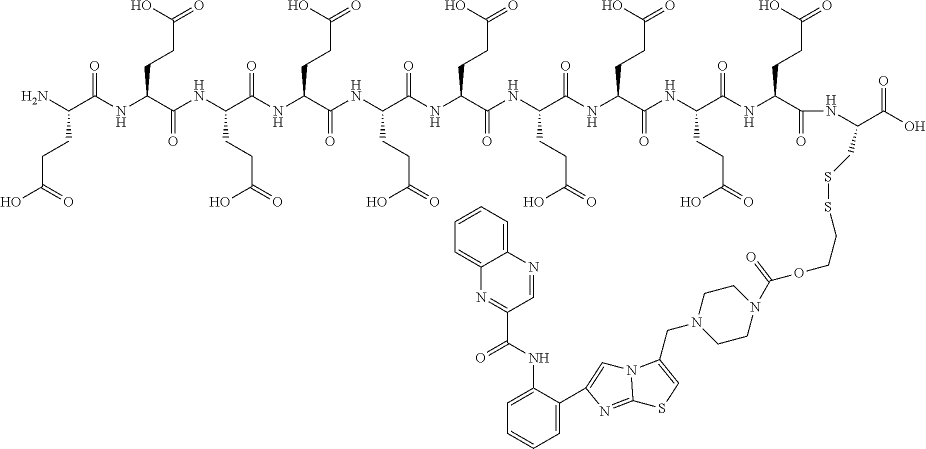 [SMILES: N[C@@H](CCC(=O)O)C(=O)N[C@@H](CCC(=O)O)C(=O)N[C@@H](CCC(=O)O)C(=O)N[C@@H](CCC(=O)O)C(=O)N[C@@H](CCC(=O)O)C(=O)N[C@@H](CCC(=O)O)C(=O)N[C@@H](CCC(=O)O)C(=O)N[C@@H](CCC(=O)O)C(=O)N[C@@H](CCC(=O)O)C(=O)N[C@@H](CCC(=O)O)C(=O)N[C@@H](CSSCCOC(=O)N1CCN(Cc2csc3nc(-c4ccccc4NC(=O)c4cnc5ccccc5n4)cn23)CC1)C(=O)O]
or a pharmaceutically acceptable salt thereof.

12. The conjugate of claim 1, or a pharmaceutically acceptable salt thereof, wherein the conjugate of the formula I is a conjugate of the formula Z-PEGₚ-X, TMP-PEGₚ-X or AOP-PEGₚ-X, or a pharmaceutically acceptable salt thereof, wherein PEG represents a polyethylene glycol radical, AOP represents acidic oligopeptides, and p is an integer from 0 to 10.

13. The conjugate of claim 1, or a pharmaceutically acceptable salt thereof, wherein the conjugate of the formula V is a conjugate of the formula Z-PEGₚ-X—X-PEG_{d}-Z, TMP-PEGₚ-X—X-PEG_{d}-TMP or AOP-PEGₚ-X—X-PEG_{d}-AOP, or a pharmaceutically acceptable salt thereof, wherein each Z, PEG, X, Z, TMP, and AOP can be the same or different, p is an integer from 0 to 10, and d is an integer from 0 to 10.

14. The conjugate of claim 1, or a pharmaceutically acceptable salt thereof, wherein the conjugate of the formula V is a conjugate of the formula: 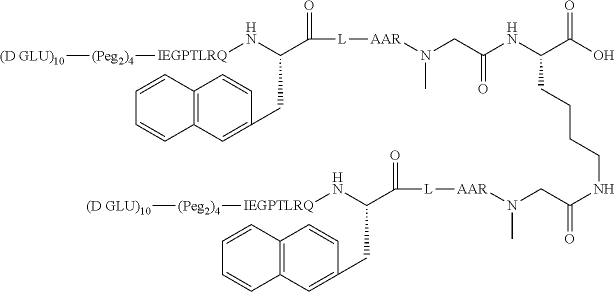
or a pharmaceutically acceptable salt thereof.

15. The conjugate of claim 1, wherein X is a sirtuin activator.

16. The conjugate of claim 15, wherein the sirtuin activator is a sirtuin 1 activator or a sirtuin 3 activator.

17. The conjugate of claim 15, wherein the sirtuin activator is a small molecule sirtuin activator.

18. The conjugate of claim 15, wherein the sirtuin activator is a selective sirtuin activator.

19. The conjugate of claim 15, wherein the sirtuin activator is a sirtuin 1 activator selected from the group consisting of SRT1720, resveratrol, forskolin, metformin, Nampt activators, AMPK activators, NMN, NAD+, STAC-5, STAC-8, STAC-9, SRT-2183, SRT1460, SRT2104, SRT3025, oxazolo[4,5-b]pyridines, pyrrolo[3,2-b]quinoxalins, and combinations thereof.

20. The conjugate of claim 1, or a pharmaceutically acceptable salt thereof wherein the conjugate of the formula II is a conjugate of the formula X-Z, where X is a radical of a molecule comprising a sirtuin activator and Z is a molecule comprising a bone-targeting molecule.

21. The conjugate of claim 1, wherein Y¹ is present and is a non-releasable linker containing at least one carbon-carbon bond, amide bond, carbon-oxygen bond, and/or carbon-sulfur bond.

22. The conjugate of claim 1, wherein Y¹ is present and is a releasable linker containing at least one disulfide (S—S), at least one ester (O(C═O)), and/or at least one protease-specific amide bond.

23. The conjugate of claim 1, wherein Y¹ comprises (PEG2)_{q}, wherein q is an integer of at least one.

24. The conjugate of claim 1, wherein X—X is a dimer of AF12505 (IEGPTLRQWLAARA) or a modified dimer of AF12505, Y¹ comprises (PEG₂)₄, and Z comprises ten D-glutamic acids.

25. The conjugate of claim 24, wherein the dimer of AF12505 is AF13948, AF13948 in which tryptophan has been substituted with naphthalene and alanine has been substituted with sarcosine, or GW395058.

26. A pharmaceutical composition comprising a therapeutically effective amount of a conjugate of claim 1 and a pharmaceutically acceptable carrier or excipient.

27. A method of treating a bone defect in a patient, which method comprises administering to the patient a therapeutically effective amount of a conjugate of claim 1 or a pharmaceutical composition comprising said conjugate, whereupon the bone fracture in the patient is treated.

28. The method of claim 27, wherein administering is injecting.

29. The method of claim 28, wherein injecting is intraperitoneally, parenterally, intramuscularly, and subcutaneously.

30. A method of treating a bone defect in a patient, which method comprises administering to the patient a therapeutically effective amount of a thrombopoietin mimetic peptide (TMP), a dimer of TMP, or a pharmaceutical composition comprising same, whereupon the bone fracture in the patient is treated.

31. The method of claim 30, wherein the TMP or dimer thereof is AF12505 (IEGPTLRQWLAARA), a modified dimer of AF12505, AF13948, AF13948 in which tryptophan has been substituted with naphthalene and alanine has been substituted with sarcosine, or GW395058 (dimer of AF12505).

32. The method of claim 31, wherein administering is injecting.

33. The method of claim 32, wherein injecting is intraperitoneally, parenterally, intramuscularly or subcutaneously.
